(19) United States
(12) Patent Application Publication
(10) Pub. No.: US 2023/0192626 A1
(43) Pub. Date: Jun. 22, 2023

(54) NOVEL SOLUBLE EPOXIDE HYDROLASE INHIBITORS AND METHOD OF USE THEREOF

(71) Applicant: Baylor College of Medicine, Houston, TX (US)

(72) Inventors: Jin Wang, Houston, TX (US); Hui Zheng, Houston, TX (US); Lisheng Deng, Houston, TX (US); Anamitra Ghosh, Houston, TX (US)

(73) Assignee: Baylor College of Medicine, Houston, TX (US)

(21) Appl. No.: 17/999,408

(22) PCT Filed: May 26, 2021
(86) PCT No.: PCT/US2021/034128

§ 371 Date: Nov. 21, 2022

Related U.S. Application Data
(60) Provisional application No. 63/030,085, filed on May 26, 2020.

(51) Int. Cl. C07D 235/18; C07D 235/14; C07D 235/30; C07D 235/26; C07D 235/20; C07D 401/04; C07D 403/04; C07D 405/04; C07D 417/04; C07D 413/10; C07D 401/10; C07D 403/10; C07D 401/12; C07D 403/12; C07D 487/04; C07D 413/12; C07D 471/04; C07D 473/40; C07D 473/34; A61K 45/06

(52) U.S. Cl. CPC C07D 235/18; C07D 235/14; C07D 235/30; C07D 235/26; C07D 235/20; C07D 401/04; C07D 403/04; C07D 405/04; C07D 417/04; C07D 413/10; C07D 401/10; C07D 403/10; C07D 401/12; C07D 403/12; C07D 487/04; C07D 413/12; C07D 471/04; C07D 473/40; C07D 473/34; A61K 45/06

(57) ABSTRACT

Novel soluble epoxide hydrolase (sEH) inhibitors are provided, along with methods for their use. The soluble epoxide hydrolase inhibitors are useful in treating and/or preventing sEH-related related diseases, such as Alzheimer's disease and inflammation. Also provided are methods for inhibiting soluble epoxide hydrolase in a cell using the compounds and compositions described herein.

### CROSS-REFERENCE TO PRIORITY APPLICATION

This application claims priority to U.S. Provisional Application No. 63/030,085, filed May 26, 2020, which is incorporated herein by reference in its entirety.

### STATEMENT REGARDING FEDERALLY FUNDED RESEARCH

This invention was made with government support under Grant No. 1RF1AG062257, awarded by the National Institutes of Health. The government has certain rights in the invention.

### BACKGROUND

Alzheimer's disease (AD) is the leading cause of dementia in the aging population and one of the leading causes of death in the United States. AD is the only leading cause of death from which no disease-modifying therapy is current available, posing an enormous socioeconomic burden. Current therapies for AD, such as acetylcholinesterase inhibitors and N-methyl-D-aspartic acid receptor antagonists, only provide symptomatic relief but fail to prevent or delay progression of the disease.

### SUMMARY

Described herein are small molecule inhibitors of soluble epoxide hydrolase (sEH) and methods for their use as neuroprotective and/or anti-inflammatory agents. The small molecule soluble epoxide hydrolase inhibitors (sEHIs) described herein are useful in treating and/or preventing inflammation and neurodegenerative disorders, such as Alzheimer's disease (AD), Parkinson's disease, dementia, cerebral ischemia, a seizure, traumatic brain injury, stroke, and other indications as described herein.

Small molecule sEH inhibitors include compounds of the following formula:

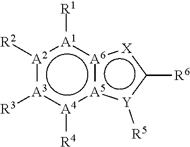

and pharmaceutically acceptable salts or prodrugs thereof. In these compounds, A¹, A², A³, A⁴, A⁵, and A⁶ are each independently selected from the group consisting of N and C; R¹, R², R³, and R⁴ are each independently selected from the group consisting of hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof; R⁵ is hydrogen, deuterium, tritium, halogen, cyano, trifluoromethyl, alkoxy, cycloalkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl, or an isotopic substitution thereof; R⁶ is hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl, or an isotopic substitution thereof; X is selected from the group consisting from N and CR⁷, wherein R⁷ is hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl, or an isotopic substitution thereof; and Y is N, O, or S, wherein when A¹, A², A³, or A⁴ is N, then the directly bonded substituent selected from R¹, R², R³, or R⁴, respectively, is absent; and wherein when Y is O or S, R⁵ is absent.

Optionally, the compound has one of the following formulas:

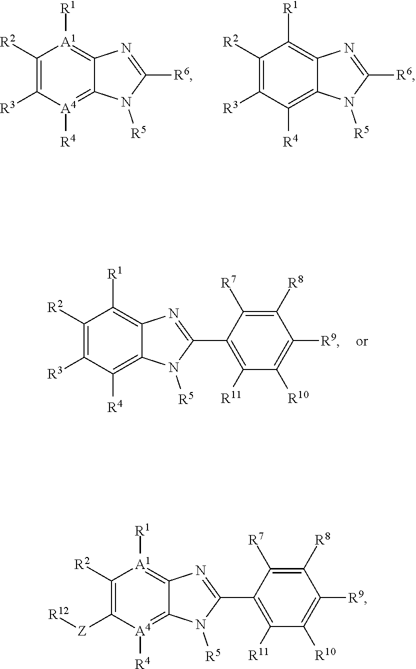

wherein

R⁷, R⁸, R⁹, R¹⁰, R¹¹, and R¹² are each independently selected from the group consisting of hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof; and

Z is CR¹³R¹⁴, NR¹³, O, S, or SO₂, wherein R¹³ and R¹⁴ are each independently selected from the group consisting of hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof.

Optionally, R¹² or R¹³ is a substituted aryl or substituted heteroaryl. Optionally, R¹² or R¹³ is

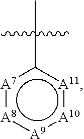

A⁷, A⁸, A⁹, A¹⁰, and A¹¹ are each independently selected from CR, N, N—OR, S, and O, and wherein each R is independently hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof. Optionally, R¹² or R¹³ is

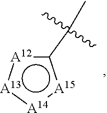

wherein
A¹², A¹³, A¹⁴, and A¹⁵ are each independently selected from CR, N, N—OR, S, and O, wherein each R is independently hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof.

Optionally, the compound has one of the following formulas:

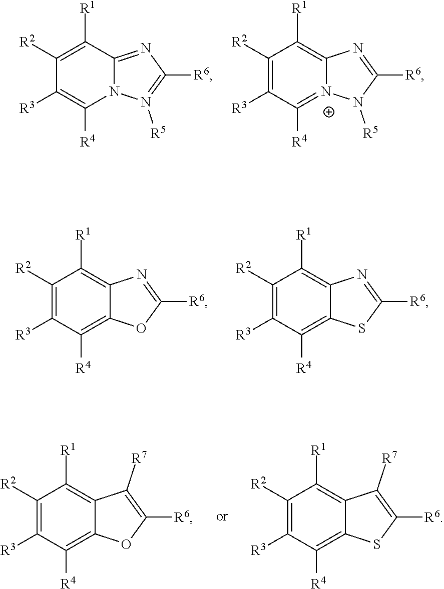

Optionally, the compound is selected from the group consisting of:

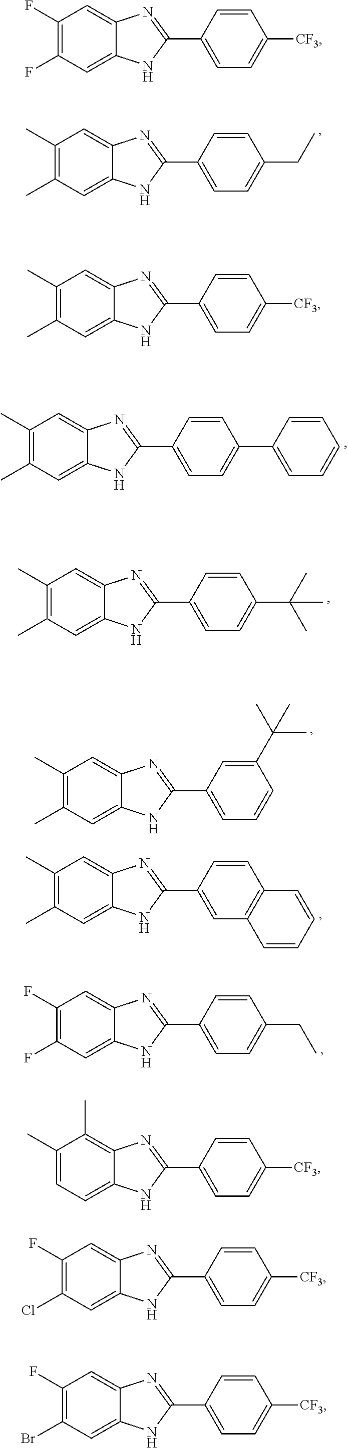

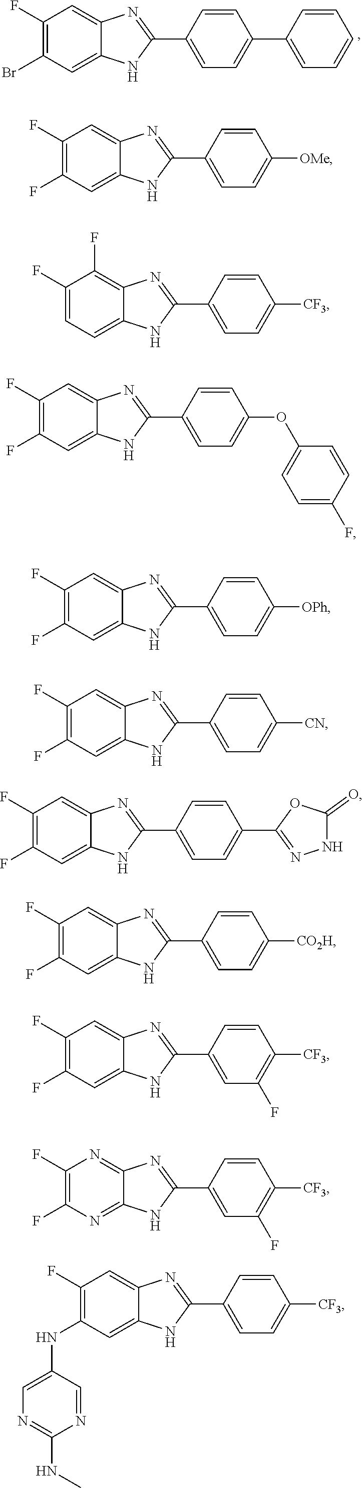

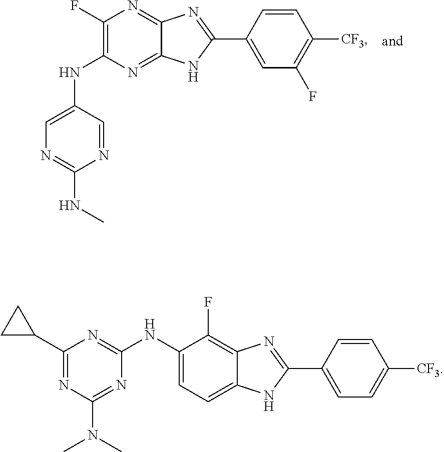 [SMILES: CNc1ncc(Nc2nc3[nH]c(-c4ccc(C(F)(F)F)c(F)c4)nc3nc2F)cn1 ; "and" ; CN(C)c1nc(Nc2ccc3[nH]c(-c4ccc(C(F)(F)F)cc4)nc3c2F)nc(C2CC2)n1]

Also described herein is a pharmaceutical composition comprising a compound as described herein and a pharmaceutically acceptable carrier.

Further described herein is a kit comprising a compound or composition as described herein.

Methods of treating or preventing a soluble epoxide hydrolase (sEH)-related disease in a subject are also provided herein. A method of treating or preventing a sEH-related disease in a subject comprises administering to the subject an effective amount of a compound or pharmaceutical composition as described above. Optionally, the sEH-related disease is a neurodegenerative disorder (e.g., Alzheimer's disease, Parkinson's disease, dementia, cerebral ischemia, a seizure, traumatic brain injury, stroke, Alexander disease, Alper's disease, amyotrophic lateral sclerosis, ataxia telangiectasia, Batten disease, Canavan disease, Cockayne syndrome, Corticobasal degeneration, Creutzfeldt-Jakob disease, Huntington's disease, Kennedy's disease, Krabbe disease, Lewy body dementia, Machado-Joseph disease, multiple sclerosis, multiple system atrophy, Pelizaeus-Merzbacher disease, Pick's disease, primary lateral sclerosis, Refsum's disease, Sandhoff disease, Schilder's disease, Spielmeyer-Vogt-Sjogren-Batten disease, spinocerebellar ataxia, spinal muscular atrophy, Steele-Richardson-Olszewski disease, Tay-Sachs, transmissible spongiform encephalopathies (TSE), or Tabes dorsalis). Optionally, the sEH-related disease is inflammation (e.g., neuroinflammation, asthma, chronic obstructive pulmonary disorder (COPD), chronic bronchitis, cystic fibrosis, atherosclerosis, post-angioplasty, restenosis, coronary artery diseases, angina, rheumatoid arthritis, osteoarthritis, dermatitis, eczematous dermatitis, psoriasis, post transplantation late and chronic solid organ rejection, systemic lupus erythematosus, dermatomyositis, polymyositis, Sjogren's syndrome, polymyalgia rheumatica, temporal arteritis, Behcet's disease, Guillain Barre syndrome, Wegener's granulomatosis, polyarteritis nodosa, an inflammatory neuropathy, vasculitis, an inflammatory disorder of adipose tissue, Kaposi's sarcoma, or a smooth muscle cell proliferative disorder). Optionally, the soluble epoxide hydrolase-related disease is hypertension, adult respiratory distress syndrome, end stage renal disease, heart failure, renal failure, liver failure, cardiac fibrosis, renal fibrosis, ischemic limb disease, intermittent claudication, endothelial dysfunction, erectile dysfunction, Raynaud's disease, a diabetic vasculopathy, an atherothrombotic disorder, or a metabolic disorder.

The methods can further comprise administering the compound or the pharmaceutical composition as described herein orally, intraperitoneally, sublingually, subcutaneously, intravenously, or any clinically acceptable administration route. Optionally, the methods further comprise administering to the subject a second compound, biomolecule, or composition. Optionally, the second compound, biomolecule, or composition is an anti-inflammatory agent (e.g., epoxyeicosatrienoic acid).

The methods can further comprise selecting a subject having an amyloid- and/or tau-biomarker. Optionally, the methods can further comprise selecting an elderly subject.

Methods of reducing amyloid beta plaque formation in a subject are also provided herein. A method of reducing amyloid beta plaque formation in a subject includes administering to a subject an effect amount of a compound or pharmaceutical composition as described herein.

Methods of inhibiting soluble epoxide hydrolase in a cell are also provided herein. A method of inhibiting soluble epoxide hydrolase in a cell includes contacting a cell with an effective amount of a compound or pharmaceutical composition as described herein. Optionally, the contacting can be performed in vitro or in vivo.

The details of one or more embodiments are set forth in the drawing and the description below. Other features, objects, and advantages will be apparent from the description and drawing, and from the claims.

### DESCRIPTION OF THE DRAWING

FIG. 1 is a graph showing the cellular activity of soluble epoxide hydrolase inhibitors as described herein.

### DETAILED DESCRIPTION

Alzheimer's disease (AD) is a progressive neurodegenerative disorder that is characterized by the formation of extracellular aggregates of amyloid beta (Aβ) peptide, fibrillary tangle of intracellular tau, and neuroinflammation. Pharmacological inhibition of soluble epoxide hydrolase (sEH) decreases the inflammatory response and reduces plaque pathology in preclinical models of AD and therefore represents a powerful therapeutic strategy for targeting multiple mechanisms of AD.

Described herein are small molecule inhibitors of soluble epoxide hydrolase (sEH) and methods for their use as neuroprotective and/or anti-inflammatory agents. Due to the high level of permeability through the blood brain barrier (BBB), the small molecule soluble epoxide hydrolase inhibitors (sEHIs) described herein are effective not only for non-central nervous system indications (e.g., hypertension, cardiovascular diseases), but also for CNS indications (e.g., neurodegenerative disorders) and for inflammation.

I. Compounds

A class of sEH inhibitors described herein is represented by Formula I:

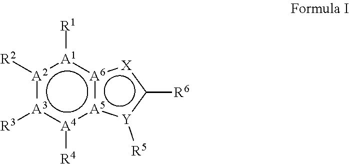

and pharmaceutically acceptable salts or prodrugs thereof.

In Formula I, A¹, A², A³, A⁴, A⁵, and A⁶ are each independently selected from the group consisting of N and C.

Also in Formula I, R¹, R², R³, and R⁴ are each independently selected from the group consisting of hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof. Optionally, R² and R³ are selected from hydrogen, fluoro, chloro, bromo, methyl, tert-butyl, and methoxy.

Additionally in Formula I, R⁵ is hydrogen, deuterium, tritium, halogen, cyano, trifluoromethyl, alkoxy, cycloalkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl, or an isotopic substitution thereof.

Further in Formula I, R⁶ is hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl, or an isotopic substitution thereof.

Also in Formula I, X is selected from the group consisting from N and CR⁷, wherein R⁷ is hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl, or an isotopic substitution thereof.

Further in Formula I, Y is N, O, or S.

In Formula I, when A¹, A², A³, or A⁴ is N, then the directly bonded substituent selected from R¹, R², R³, or R⁴, respectively, is absent. In other words, when A¹ is N, then R¹ is absent; when A² is N, then R² is absent, when A³ is N, then R³ is absent; and when A⁴ is N, then R⁴ is absent.

Also in Formula I, when Y is O or S, R⁵ is absent.

Optionally, adjacent R groups in Formula I, e.g., R¹ and R², R² and R³, R³ and R⁴, R⁵ and R⁶, or R⁶ and R⁷ can be combined to form a substituted or unsubstituted aryl, substituted or unsubstituted cycloalkenyl, substituted or unsubstituted heteroaryl, or substituted or unsubstituted heterocycloalkyl.

In some cases, the compounds according to Formula I are represented by Structure I-A:

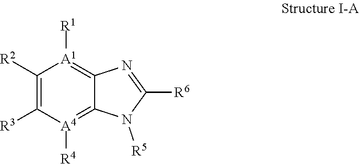

In Structure I-A, A¹, A⁴, R¹, R², R³, R⁴, R⁵, and R⁶ are as defined above for Formula I. In Structure I-A when A¹ or A⁴ is N, then the directly bonded substituent selected from R¹ or R, respectively, is absent. In other words, when A¹ is N, then R¹ is absent and when A⁴ is N, then R⁴ is absent.

In some cases of the compounds according to Structure I-A, A¹ and A⁴ are both C. In other words, in some cases, the compounds according to Structure I-A are represented by Structure I-B:

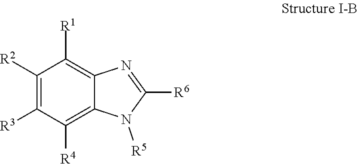

In Structure I-B, R¹, R², R³, R⁴, R⁵, and R⁶ are as defined above for Formula I.

In some cases of the compounds according to Structure I-B, R⁵ is substituted or unsubstituted aryl or substituted or unsubstituted heteroaryl. In some cases, the compounds according to Structure I-B are represented by Structure I-C, as shown below:

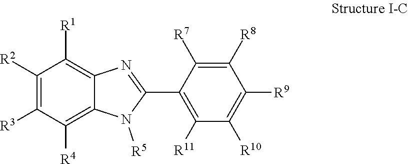

In Structure I-C, R¹, R², R³, R⁴, and R⁵ are as defined above for Formula I. Also in Structure I-C, R⁷, R⁸, R⁹, R¹⁰, and R¹¹ are each independently selected from the group consisting of hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof. Optionally, at least one of R¹, R², R³, R⁴, R⁷, R⁸, R⁹, R¹⁰, and R¹¹ is a halogen (e.g., Cl, F, Br, or I) or contains a halogen (e.g., halogen substituted alkyl or halogen substituted aryl).

In some cases of the compounds according to Structure I-A, R⁶ is substituted or unsubstituted aryl or substituted or unsubstituted heteroaryl and R³ is substituted or unsubstituted amino (e.g., R³ is optionally cycloalkyl- or aryl-substituted amino), substituted or unsubstituted alkyl, alkoxy, cycloalkoxy, aryloxy, thio, or sulfonyl. Optionally, the compounds according to Structure I-A are represented by Structure 1-D:

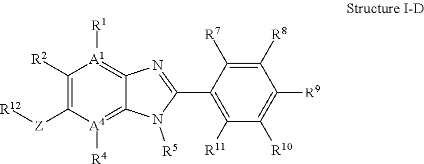

In Structure I-D, A¹, A⁴, R¹, R², R⁴, R⁵, R⁷, R⁸, R⁹, R¹⁰, and R¹¹ are as defined above. In Structure I-D when A¹ or A⁴ is N, then the directly bonded substituent selected from R¹ or R⁴, respectively, is absent. In other words, when A¹ is N, then R¹ is absent and when A⁴ is N, then R⁴ is absent. Also in Structure I-D, R¹² is hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl, or an isotopic substitution thereof. Additionally in Structure I-D, Z is CR¹³R¹⁴, NR¹³, O, S, or SO₂, wherein R¹³ and R¹⁴ are each independently selected from the group consisting of hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof.

Optionally, R¹² or R¹³ is a substituted or unsubstituted aryl or substituted or unsubstituted heteroaryl as represented below:

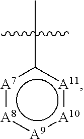

wherein A⁷, A⁸, A⁹, A¹⁰, and A¹¹ are each independently selected from CR, N, N—OR, S, and O, and wherein each R is independently hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof.

Optionally, R¹² or R¹³ is a substituted or unsubstituted aryl or substituted or unsubstituted heteroaryl as represented below:

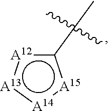

wherein A¹², A¹³, A¹⁴, and A¹⁵ are each independently selected from CR, N, N—OR, S, and O, wherein each R is independently hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof.

In some cases, the compounds according to Formula I are represented by Structure I-E:

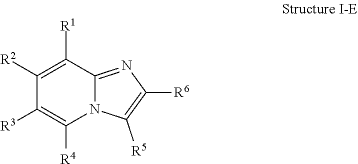

In Structure I-E, R¹, R², R³, R⁴, R⁵, and R⁶ are as defined above for Formula I.

In some cases, the compounds according to Formula I are represented by Structure I-F:

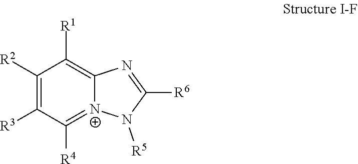

In Structure I-F, R¹, R², R³, R⁴, R⁵, and R⁶ are as defined above for Formula I.

In some cases, the compounds according to Formula I are represented by Structure I-G:

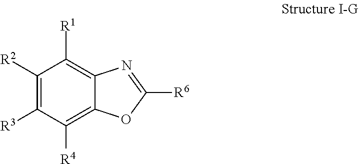

In Structure I-G, R¹, R², R³, R⁴, and R are as defined above for Formula I.

In some cases, the compounds according to Formula I are represented by Structure I-H:

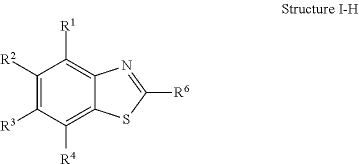

In Structure I-H, R¹, R², R³, R⁴, and R⁶ are as defined above for Formula I.

In some cases, the compounds according to Formula I are represented by Structure I-I:

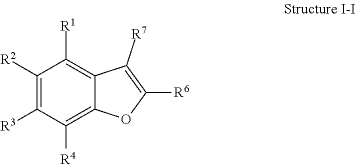

In Structure I-I, R¹, R², R³, R⁴, R⁶, and R⁷ are as defined above for Formula I.

In some cases, the compounds according to Formula I are represented by Structure I-J:

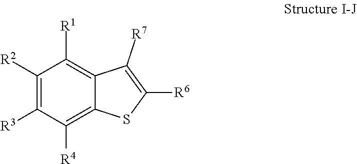

In Structure I-J, R¹, R², R³, R⁴, R⁵, and R⁶ are as defined above for Formula I.

Examples of Formula I include the following compounds:

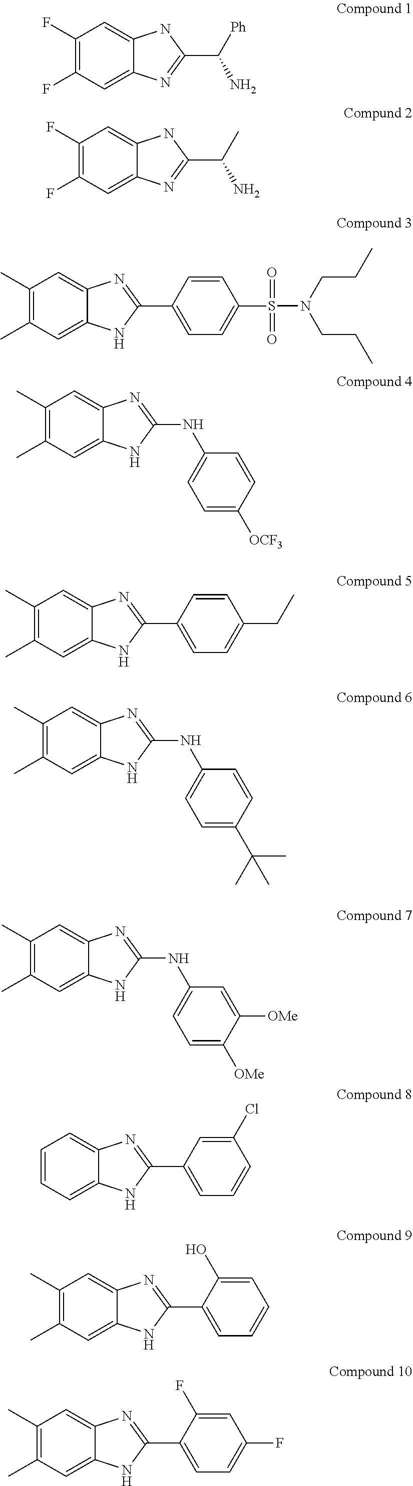

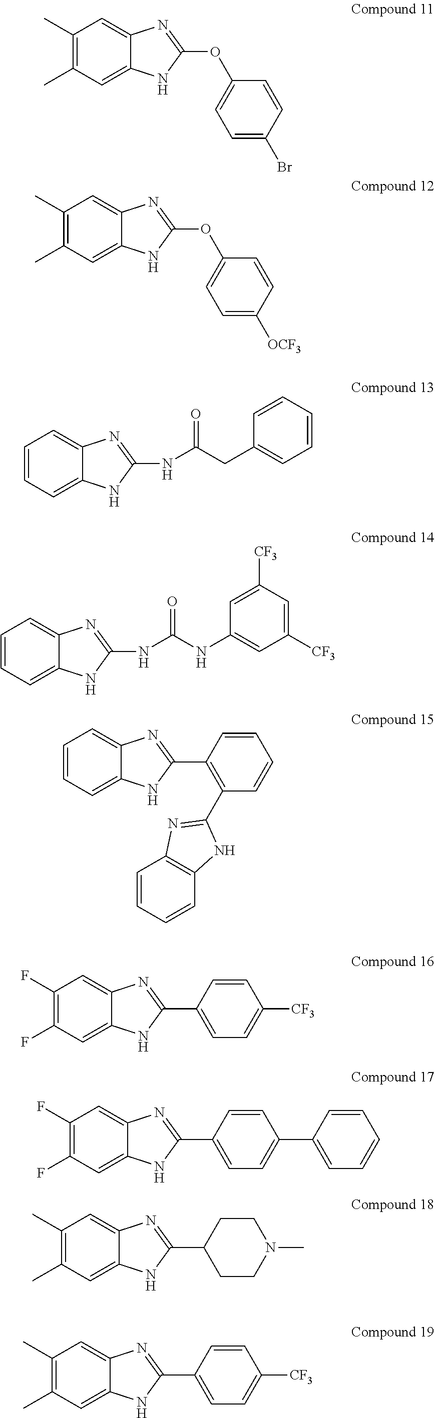

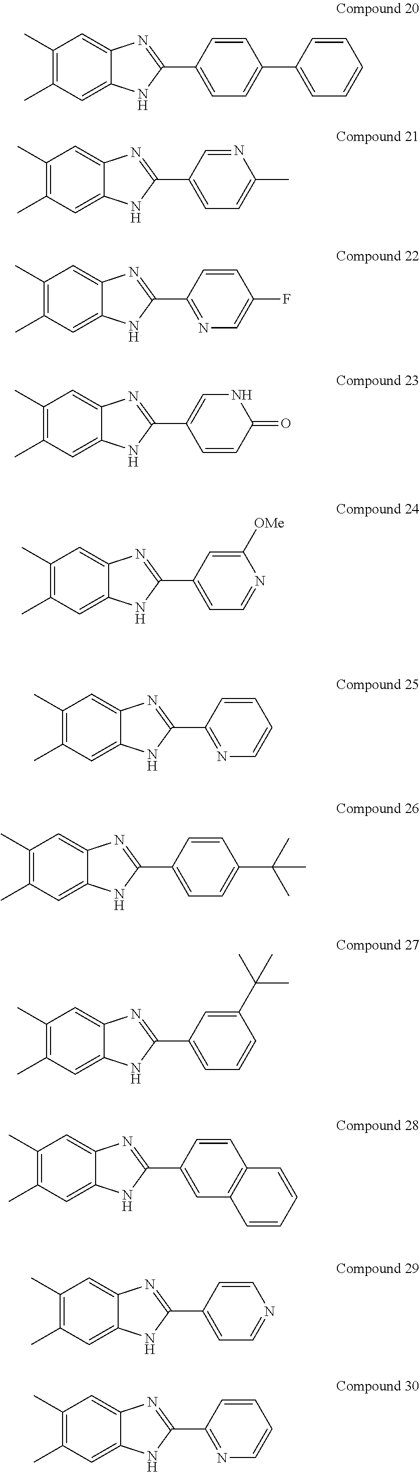

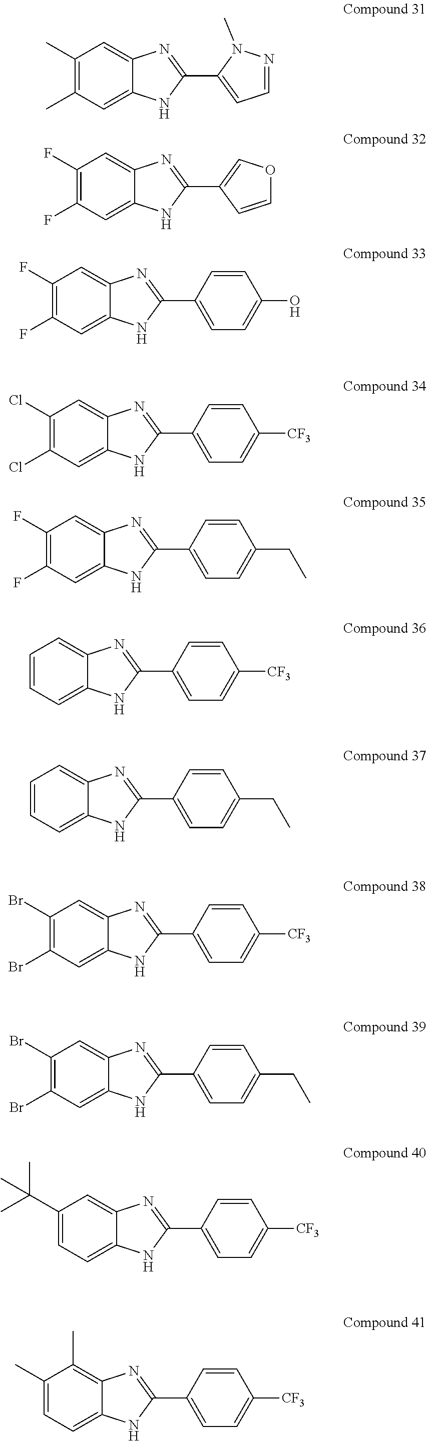

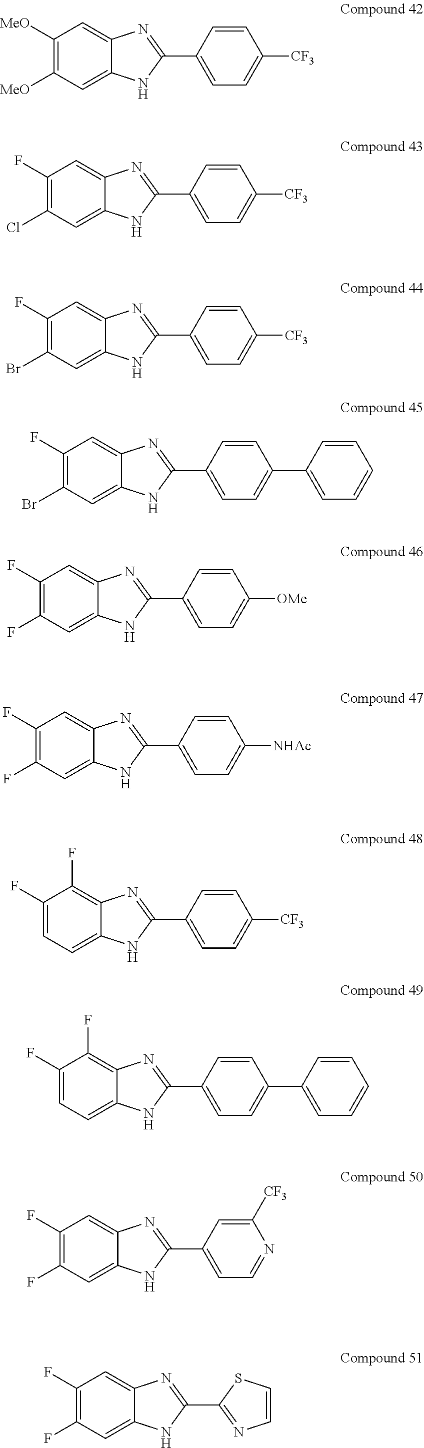

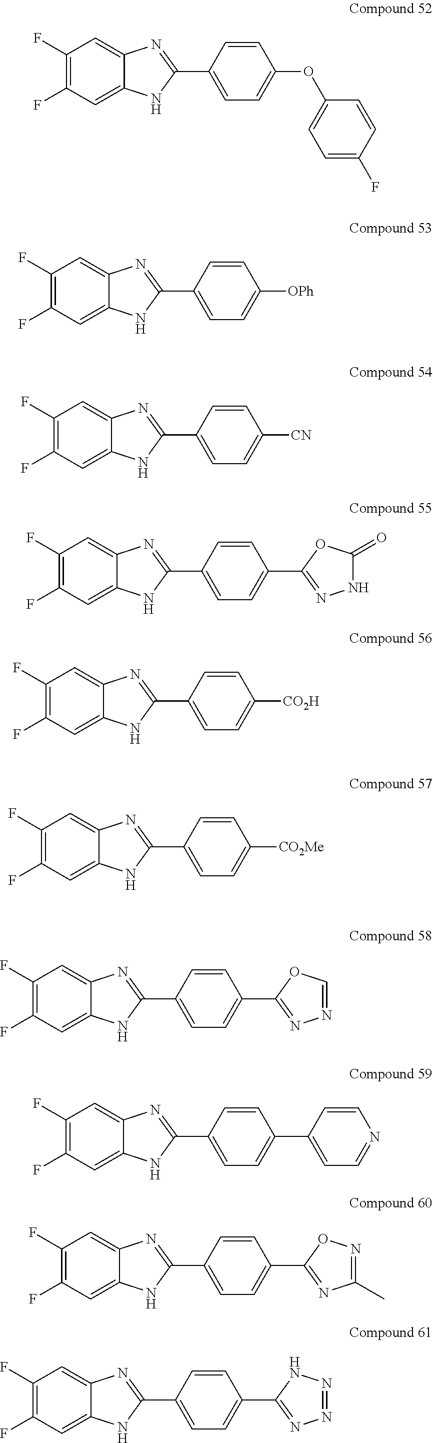

Additional examples of Formula I include the following compounds:

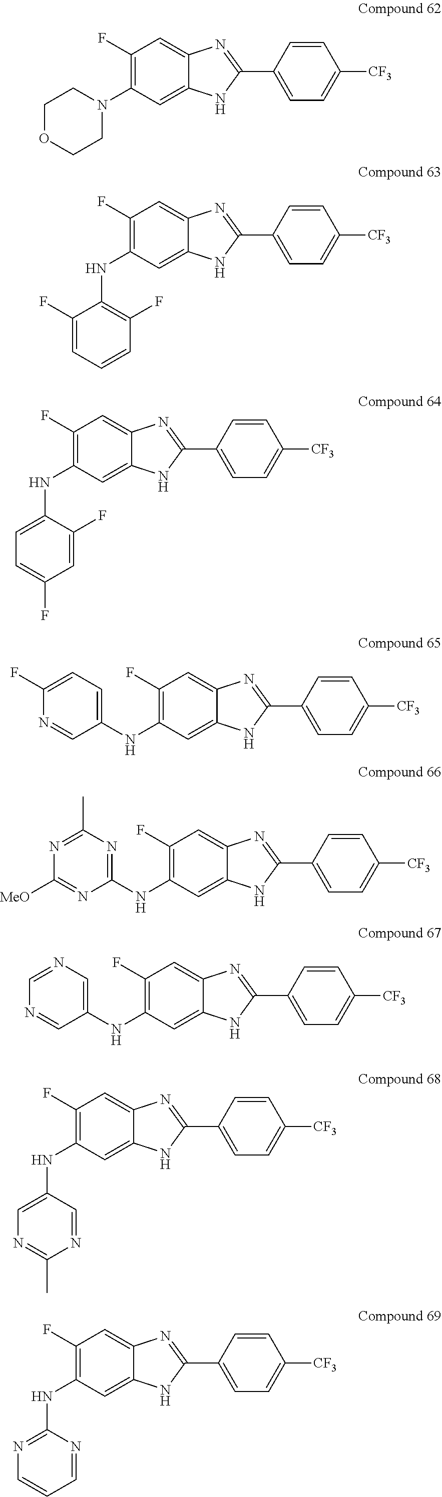

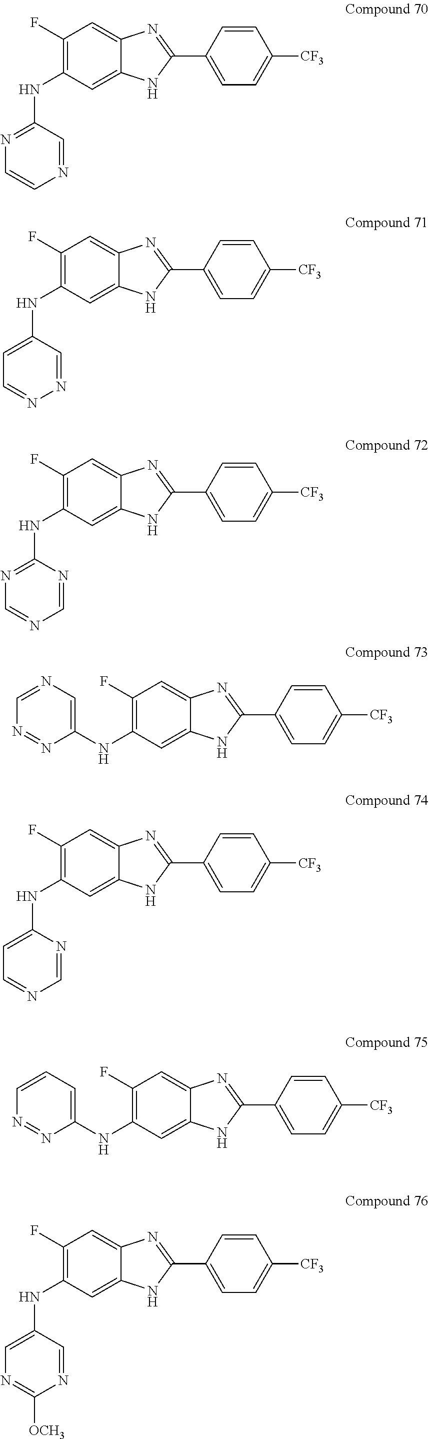

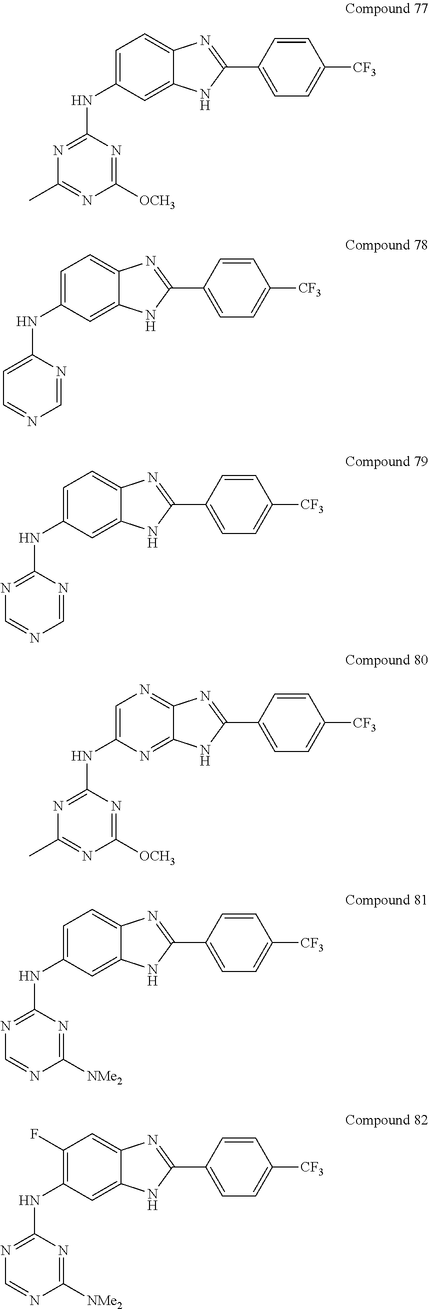 [SMILES: COc1nc(C)nc(Nc2ccc3nc(-c4ccc(C(F)(F)F)cc4)[nH]c3c2)n1 ; FC(F)(F)c1ccc(-c2nc3ccc(Nc4ccncn4)cc3[nH]2)cc1 ; FC(F)(F)c1ccc(-c2nc3ccc(Nc4ncncn4)cc3[nH]2)cc1 ; COc1nc(C)nc(Nc2cnc3nc(-c4ccc(C(F)(F)F)cc4)[nH]c3n2)n1 ; CN(C)c1ncnc(Nc2ccc3nc(-c4ccc(C(F)(F)F)cc4)[nH]c3c2)n1 ; CN(C)c1ncnc(Nc2cc3[nH]c(-c4ccc(C(F)(F)F)cc4)nc3cc2F)n1]

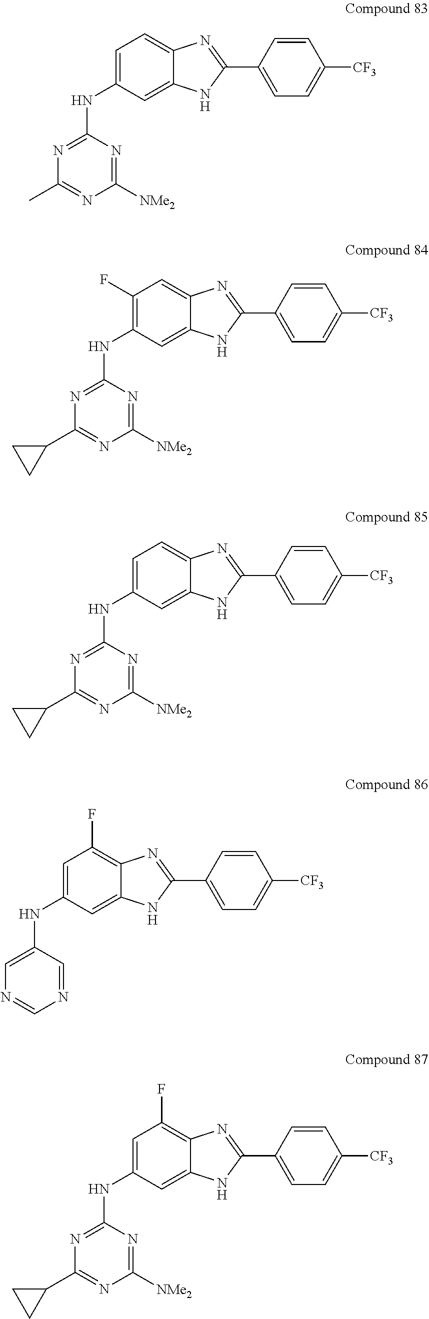 [SMILES: Cc1nc(Nc2ccc3nc(-c4ccc(C(F)(F)F)cc4)[nH]c3c2)nc(N(C)C)n1 ; CN(C)c1nc(Nc2cc3[nH]c(-c4ccc(C(F)(F)F)cc4)nc3cc2F)nc(C2CC2)n1 ; CN(C)c1nc(Nc2ccc3nc(-c4ccc(C(F)(F)F)cc4)[nH]c3c2)nc(C2CC2)n1 ; Fc1cc(Nc2cncnc2)cc2[nH]c(-c3ccc(C(F)(F)F)cc3)nc12 ; CN(C)c1nc(Nc2cc(F)c3nc(-c4ccc(C(F)(F)F)cc4)[nH]c3c2)nc(C2CC2)n1]

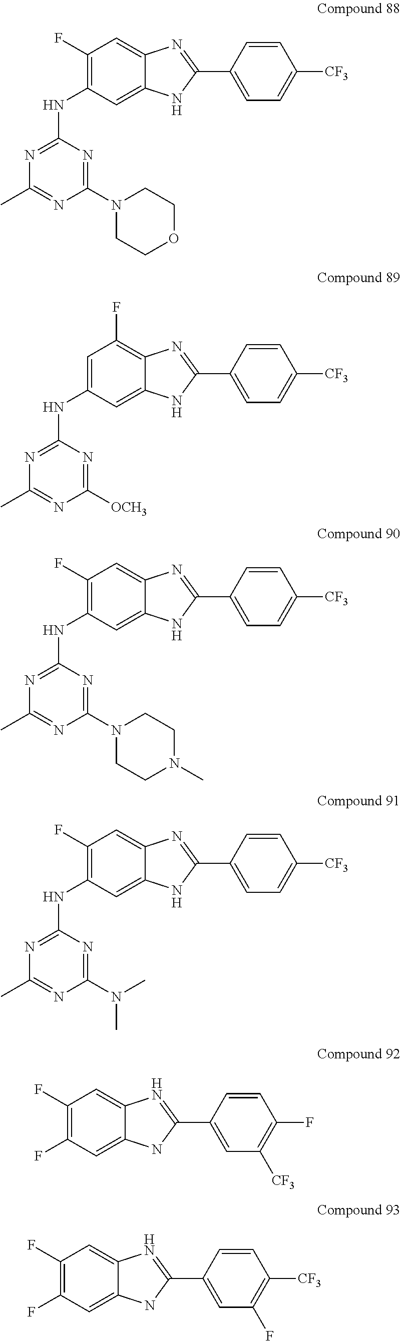

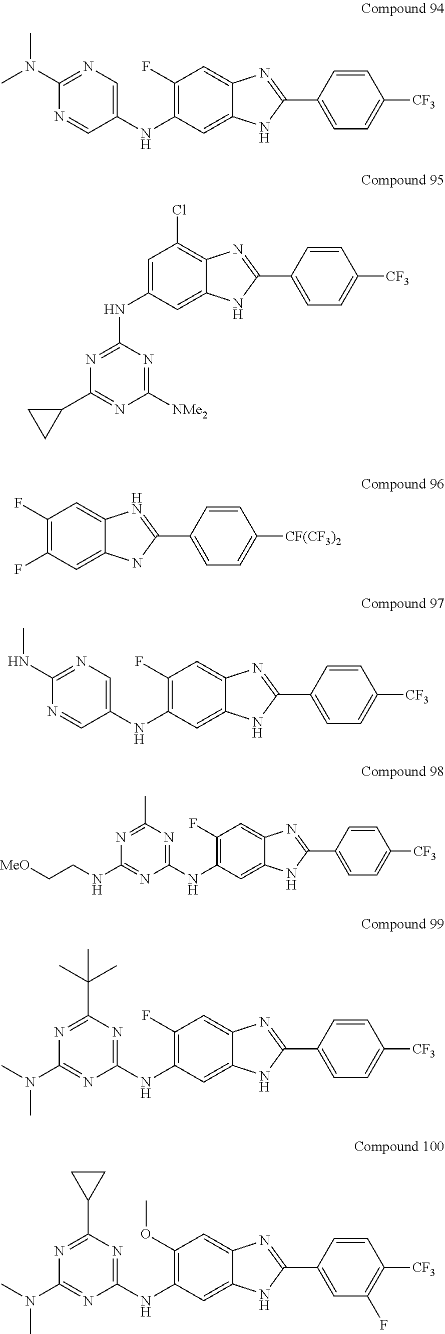

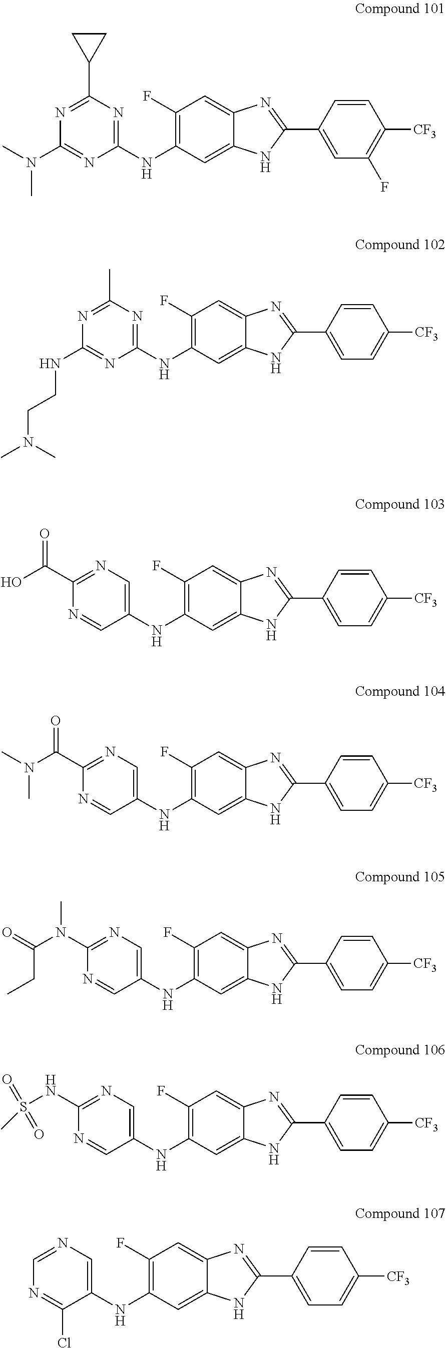

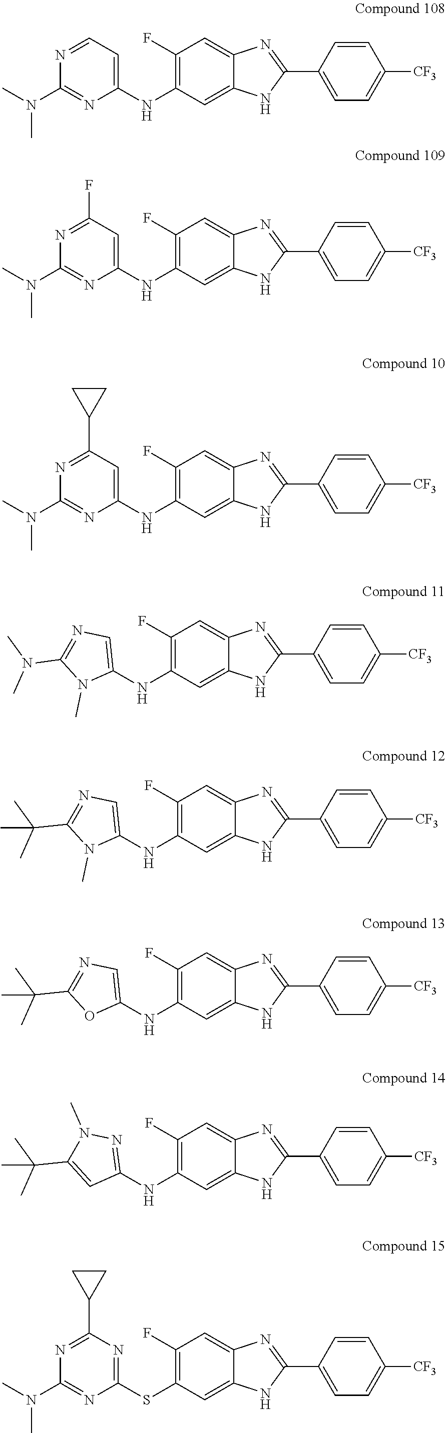

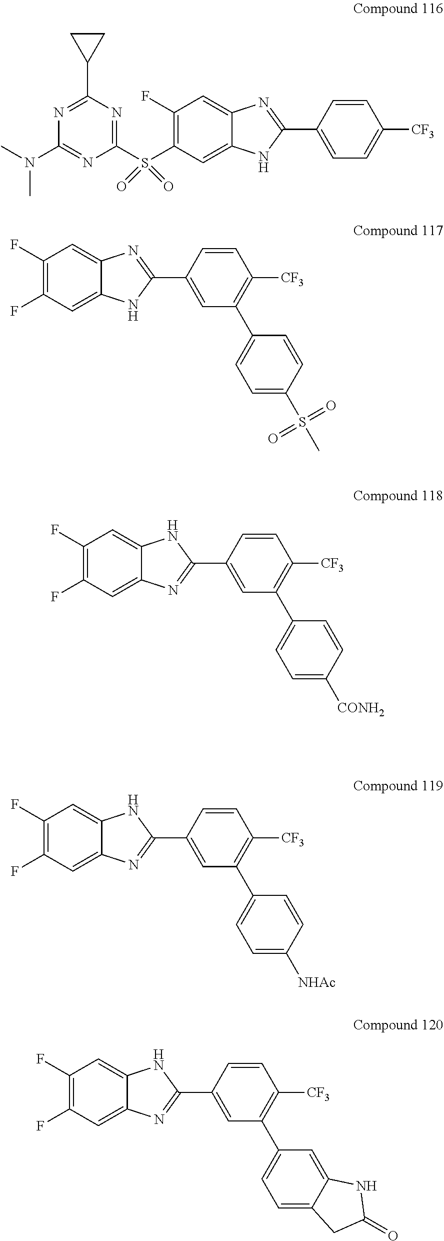 [SMILES: CN(C)c1nc(C2CC2)nc(S(=O)(=O)c2cc3[nH]c(-c4ccc(C(F)(F)F)cc4)nc3cc2F)n1 ; CS(=O)(=O)c1ccc(-c2cc(-c3nc4cc(F)c(F)cc4[nH]3)ccc2C(F)(F)F)cc1 ; NC(=O)c1ccc(-c2cc(-c3nc4cc(F)c(F)cc4[nH]3)ccc2C(F)(F)F)cc1 ; CC(=O)Nc1ccc(-c2cc(-c3nc4cc(F)c(F)cc4[nH]3)ccc2C(F)(F)F)cc1 ; O=C1Cc2ccc(-c3cc(-c4nc5cc(F)c(F)cc5[nH]4)ccc3C(F)(F)F)cc2N1]

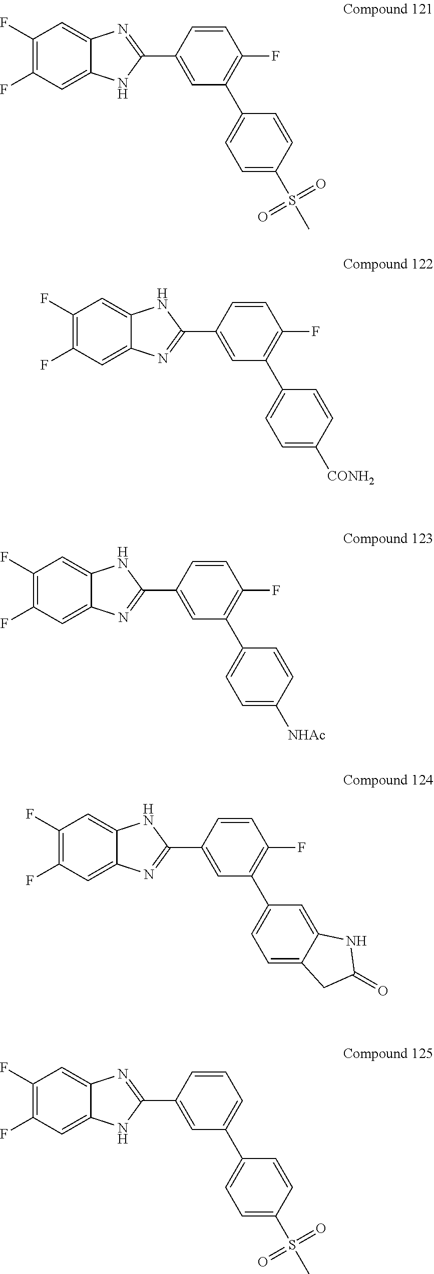 [SMILES: CS(=O)(=O)c1ccc(-c2cc(-c3nc4cc(F)c(F)cc4[nH]3)ccc2F)cc1 ; NC(=O)c1ccc(-c2cc(-c3nc4cc(F)c(F)cc4[nH]3)ccc2F)cc1 ; CC(=O)Nc1ccc(-c2cc(-c3nc4cc(F)c(F)cc4[nH]3)ccc2F)cc1 ; O=C1Cc2ccc(-c3cc(-c4nc5cc(F)c(F)cc5[nH]4)ccc3F)cc2N1 ; CS(=O)(=O)c1ccc(-c2cccc(-c3nc4cc(F)c(F)cc4[nH]3)c2)cc1]

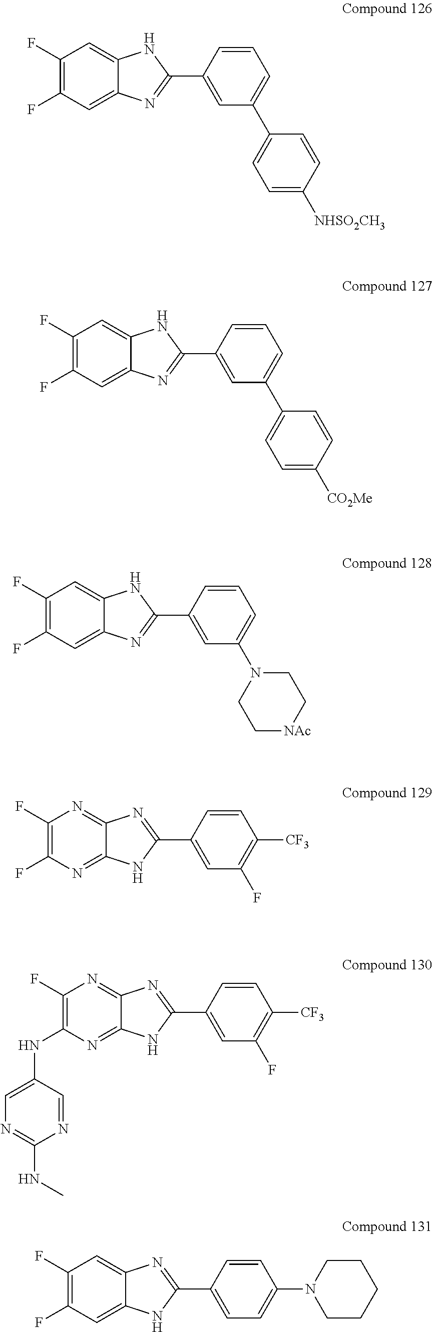

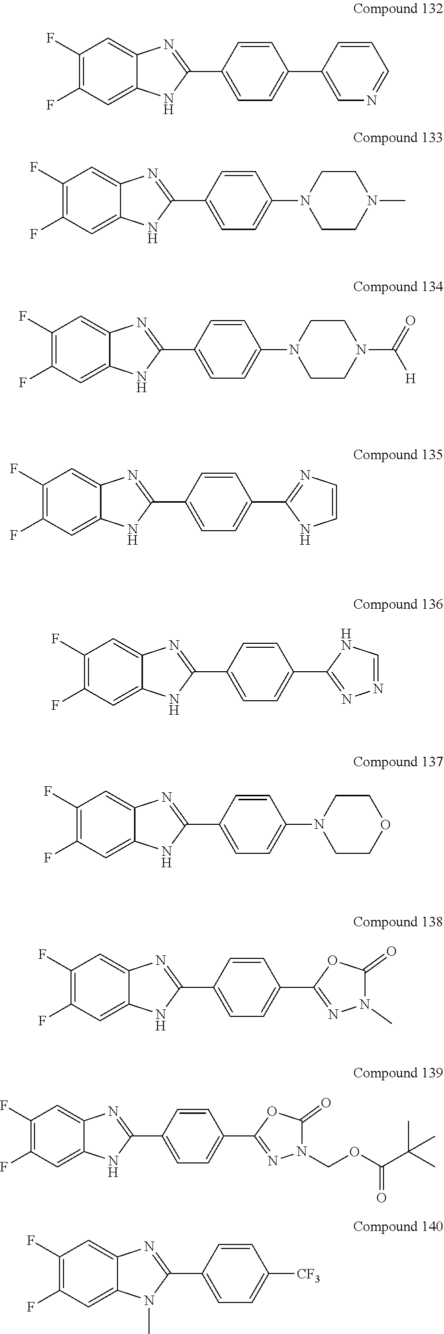

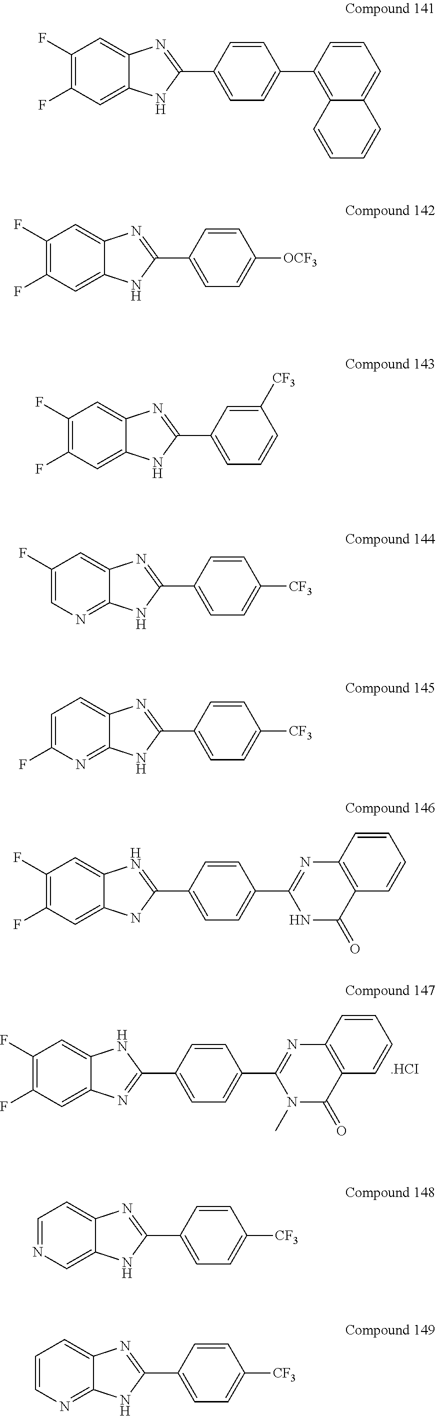

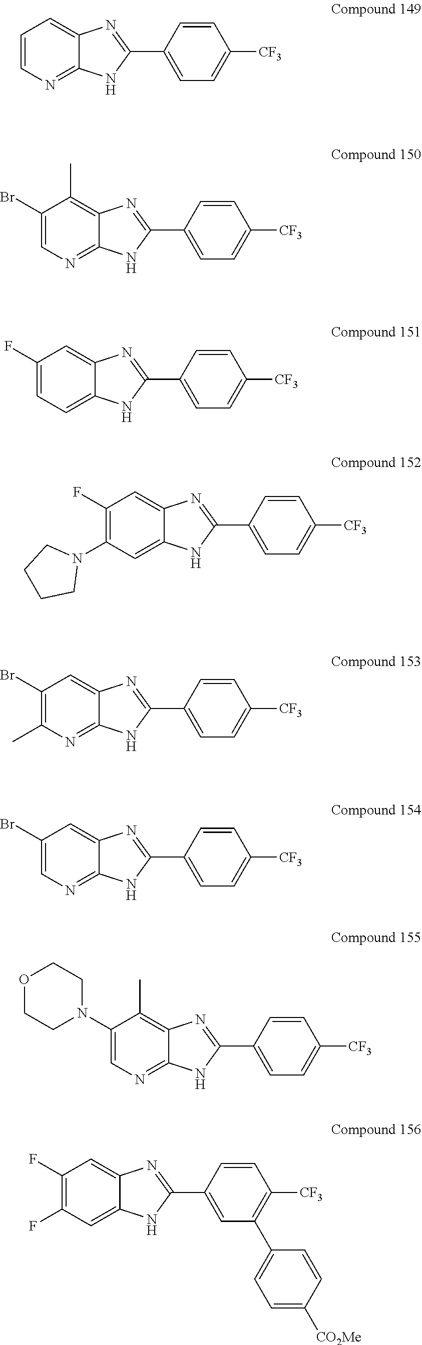

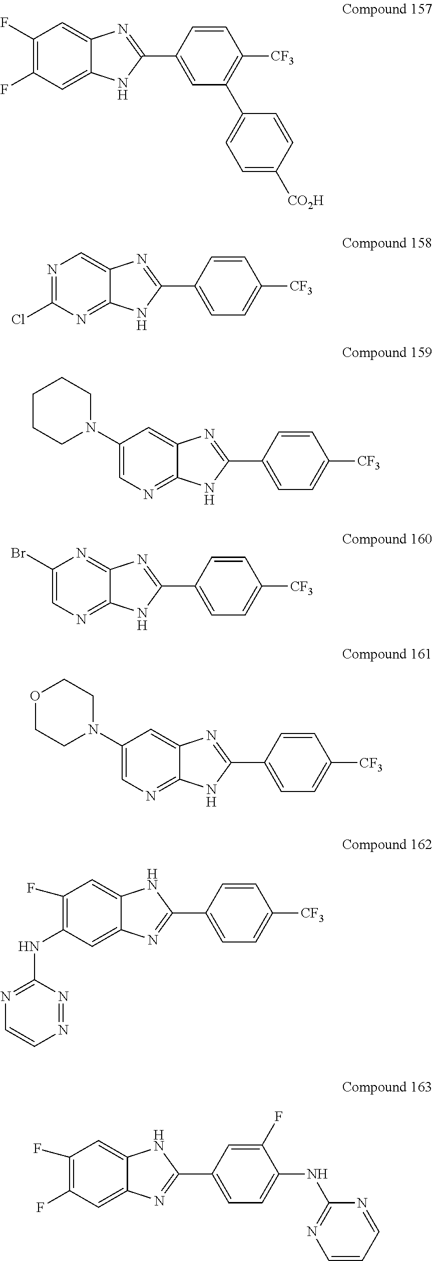

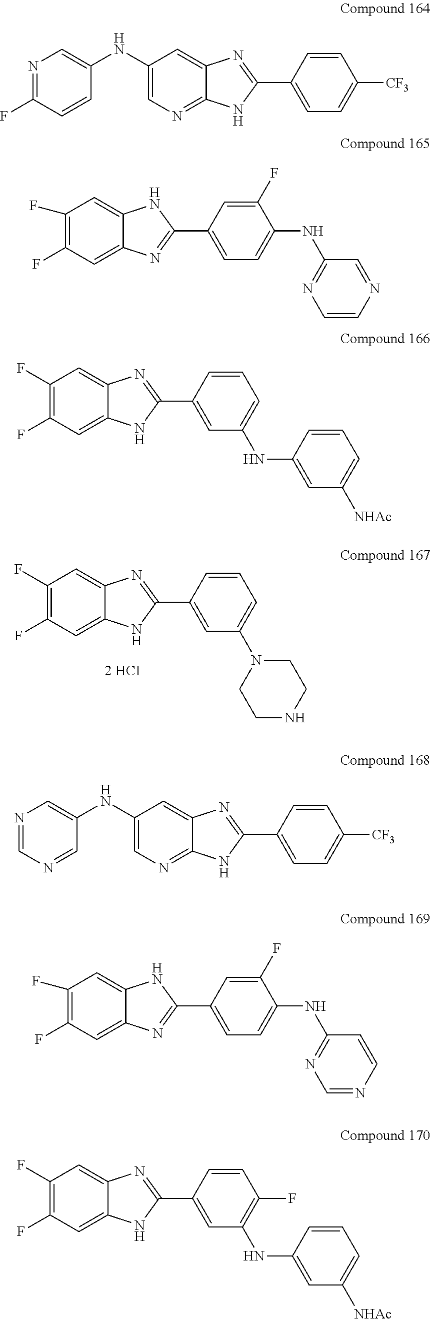

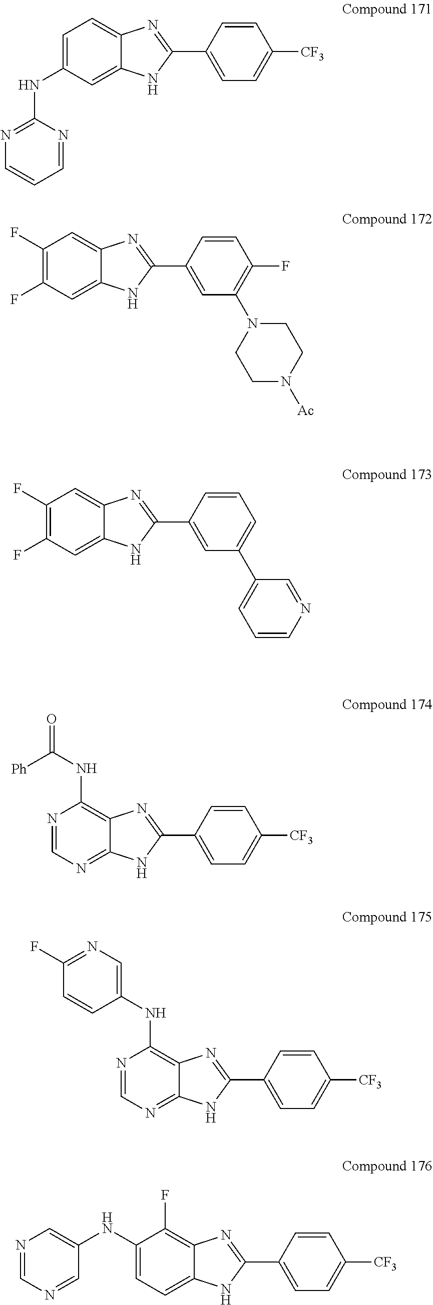 [SMILES: FC(F)(F)c1ccc(-c2nc3ccc(Nc4ncccn4)cc3[nH]2)cc1 ; CC(=O)N1CCN(c2cc(-c3nc4cc(F)c(F)cc4[nH]3)ccc2F)CC1 ; Fc1cc2nc(-c3cccc(-c4cccnc4)c3)[nH]c2cc1F ; O=C(Nc1ncnc2[nH]c(-c3ccc(C(F)(F)F)cc3)nc12)c1ccccc1 ; Fc1ccc(Nc2ncnc3[nH]c(-c4ccc(C(F)(F)F)cc4)nc23)cn1 ; Fc1c(Nc2cncnc2)ccc2[nH]c(-c3ccc(C(F)(F)F)cc3)nc12]

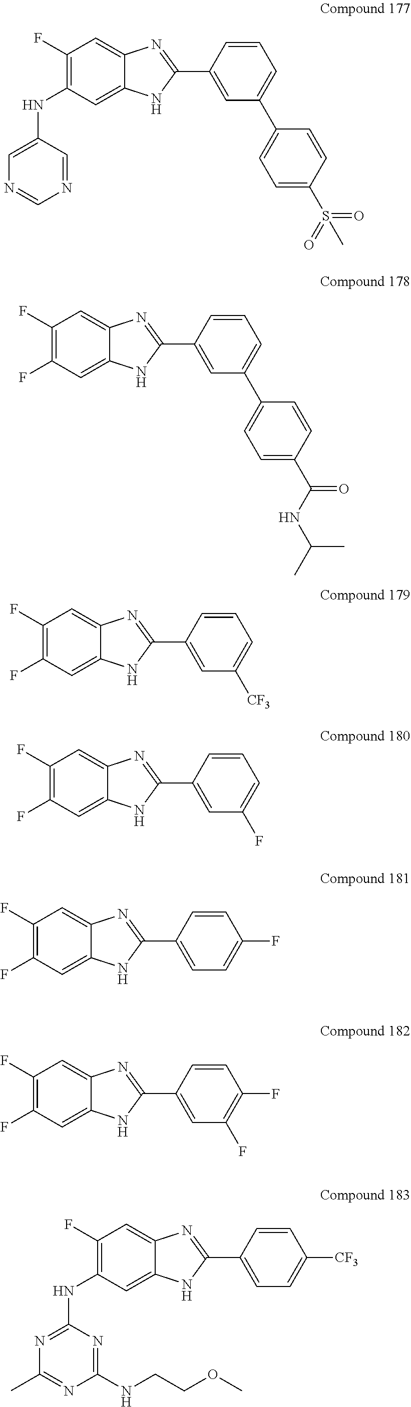

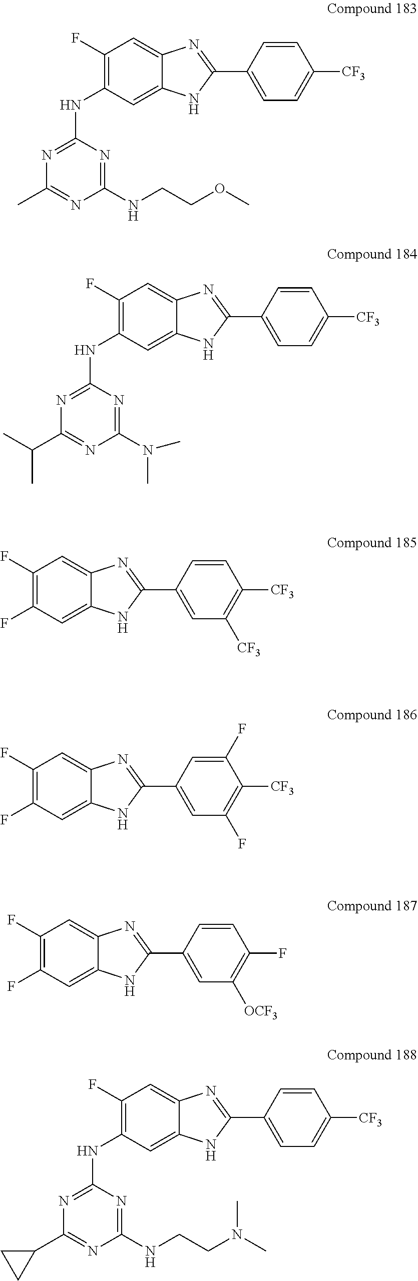 [SMILES: COCCNc1nc(C)nc(Nc2cc3[nH]c(-c4ccc(C(F)(F)F)cc4)nc3cc2F)n1 ; CC(C)c1nc(Nc2cc3[nH]c(-c4ccc(C(F)(F)F)cc4)nc3cc2F)nc(N(C)C)n1 ; Fc1cc2nc(-c3ccc(C(F)(F)F)c(C(F)(F)F)c3)[nH]c2cc1F ; Fc1cc2nc(-c3cc(F)c(C(F)(F)F)c(F)c3)[nH]c2cc1F ; Fc1cc2nc(-c3ccc(F)c(OC(F)(F)F)c3)[nH]c2cc1F ; CN(C)CCNc1nc(Nc2cc3[nH]c(-c4ccc(C(F)(F)F)cc4)nc3cc2F)nc(C2CC2)n1]

In some cases, the compound is not one or more of the following:

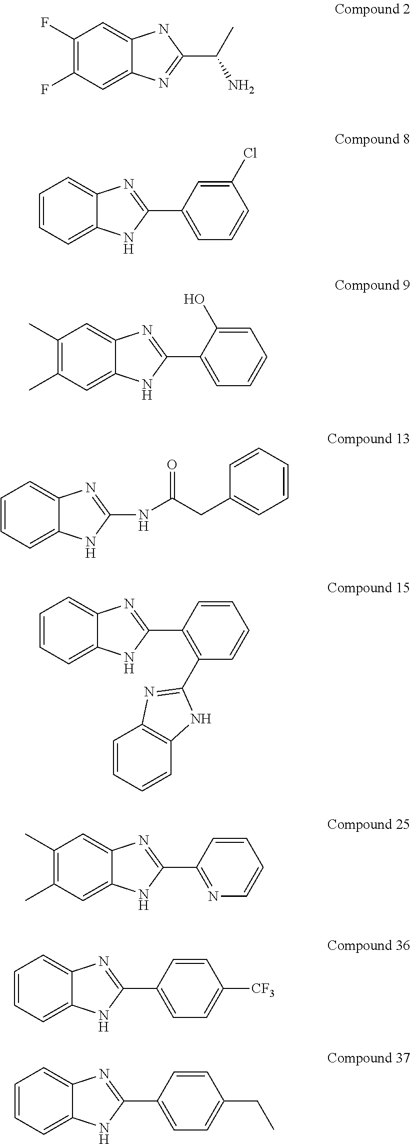

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bound through a single, terminal ether linkage. The term aryloxy as used herein is an aryl group bound through a single, terminal ether linkage. Likewise, the terms alkenyloxy, alkynyloxy, heteroalkyloxy, heteroalkenyloxy, heteroalkynyloxy, heteroaryloxy, cycloalkyloxy, and heterocycloalkyloxy as used herein are an alkenyloxy, alkynyloxy, heteroalkyloxy, heteroalkenyloxy, heteroalkynyloxy, heteroaryloxy, cycloalkyloxy, and heterocycloalkyloxy group, respectively, bound through a single, terminal ether linkage.

The term hydroxy as used herein is represented by the formula —OH.

The terms amine or amino as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl molecules used herein can be substituted or unsubstituted. As used herein, the term substituted includes the addition of an alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens, i.e., commensurate with its saturation level, with no substitutions, e.g., linear decane (—(CH₂)₉—CH₃).

II. Methods of Making the Compounds

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Variations on Formula I include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure.

Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

III. Pharmaceutical Formulations

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22d Edition, Loyd et al. eds., Pharmaceutical Press and Philadelphia College of Pharmacy at University of the Sciences (2012). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium, and/or nonionic surfactants, such as TWEEN® (ICI, Inc.; Bridgewater, N.J.), polyethylene glycol (PEG), and PLURONICS™ (BASF; Florham Park, N.J.).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, inhalants, and skin patches. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

Optionally, the compounds described herein can be contained in a drug depot. A drug depot comprises a physical structure to facilitate implantation and retention in a desired site (e.g., a synovial joint, a disc space, a spinal canal, abdominal area, a tissue of the patient, etc.). The drug depot can provide an optimal concentration gradient of the compound at a distance of up to about 0.1 cm to about 5 cm from the implant site. A depot, as used herein, includes but is not limited to capsules, microspheres, microparticles, microcapsules, microfibers particles, nanospheres, nanoparticles, coating, matrices, wafers, pills, pellets, emulsions, liposomes, micelles, gels, antibody-compound conjugates, protein-compound conjugates, or other pharmaceutical delivery compositions. Suitable materials for the depot include pharmaceutically acceptable biodegradable materials that are preferably FDA approved or GRAS materials. These materials can be polymeric or non-polymeric, as well as synthetic or naturally occurring, or a combination thereof. The depot can optionally include a drug pump.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0.0001 to about 200 mg/kg of body weight of active compound per day, which may be administered in a single dose or in the form of individual divided doses, such as from 1 to 4 times per day. Alternatively, the dosage amount can be from about 0.01 to about 150 mg/kg of body weight of active compound per day, about 0.1 to 100 mg/kg of body weight of active compound per day, about 0.5 to about 75 mg/kg of body weight of active compound per day, about 0.5 to about 50 mg/kg of body weight of active compound per day, about 0.01 to about 50 mg/kg of body weight of active compound per day, about 0.05 to about 25 mg/kg of body weight of active compound per day, about 0.1 to about 25 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, about 5 mg/kg of body weight of active compound per day, about 2.5 mg/kg of body weight of active compound per day, about 1.0 mg/kg of body weight of active compound per day, or about 0.5 mg/kg of body weight of active compound per day, or any range derivable therein. Optionally, the dosage amounts are from about 0.01 mg/kg to about 10 mg/kg of body weight of active compound per day. Optionally, the dosage amount is from about 0.01 mg/kg to about 5 mg/kg. Optionally, the dosage amount is from about 0.01 mg/kg to about 2.5 mg/kg.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

IV. Methods of Use

Provided herein are methods to treat or prevent a soluble epoxide hydrolase (sEH)-related disease in a subject. The methods include administering to a subject an effective amount of one or more of the compounds or compositions described herein, or a pharmaceutically acceptable salt or prodrug thereof. Effective amount, when used to describe an amount of compound in a method, refers to the amount of a compound that achieves the desired pharmacological effect or other biological effect. The effective amount can be, for example, the concentrations of compounds at which sEH is inhibited in vitro, as provided herein. Also contemplated is a method that includes administering to the subject an amount of one or more compounds described herein such that an in vivo concentration at a target cell in the subject corresponding to the concentration administered in vitro is achieved.

The compounds and compositions described herein or pharmaceutically acceptable salts thereof are useful for treating sEH-related diseases in humans, including, without limitation, pediatric and geriatric populations, and in animals, e.g., veterinary applications.

In some embodiments, the sEH-related disease is a neurodegenerative disorder. Optionally, the neurodegenerative disorder is Alzheimer's disease. Optionally, the neurodegenerative disorder is Parkinson's disease, dementia, cerebral ischemia, a seizure, traumatic brain injury, or stroke. Optionally, the neurodegenerative disorder is Alexander disease, Alper's disease, amyotrophic lateral sclerosis, ataxia telangiectasia, Batten disease (also known as Spielmeyer-Vogt-Sjogren-Batten disease), Canavan disease, Cockayne syndrome, Corticobasal degeneration, Creutzfeldt-Jakob disease, Huntington's disease, Kennedy's disease, Krabbe disease, Lewy body dementia, Machado-Joseph disease, multiple sclerosis, multiple system atrophy, Pelizaeus-Merzbacher disease, Pick's disease, primary lateral sclerosis, Refsum's disease, Sandhoff disease, Schilder's disease, Spielmeyer-Vogt-Sjogren-Batten disease (also known as Batten disease), spinocerebellar ataxia (multiple types with varying characteristics), spinal muscular atrophy, Steele-Richardson-Olszewski disease, Tay-Sachs, transmissible spongiform encephalopathies (TSE), or Tabes dorsalis.

In some embodiments, the sEH-related disease is inflammation. Optionally, the inflammation is neuroinflammation. Generally, inflammatory disorders include, but are not limited to, respiratory or pulmonary disorders (including asthma, chronic obstructive pulmonary disorder (COPD), chronic bronchitis and cystic fibrosis); cardiovascular related disorders (including atherosclerosis, post-angioplasty, restenosis, coronary artery diseases and angina); inflammatory diseases of the joints (including rheumatoid arthritis and osteoarthritis); skin disorders (including dermatitis, eczematous dermatitis and psoriasis); post transplantation late and chronic solid organ rejection; autoimmune conditions (including systemic lupus erythematosus, dermatomyositis, polymyositis, Sjogren's syndrome, polymyalgia rheumatica, temporal arteritis, Behcet's disease, Guillain Barre syndrome, Wegener's granulomatosis, polyarteritis nodosa); inflammatory neuropathies (including inflammatory polyneuropathies); vasculitis (including Churg-Strauss syndrome, Takayasu's arteritis); inflammatory disorders of adipose tissue; and proliferative disorders (including Kaposi's sarcoma and other proliferative disorders of smooth muscle cells).

Optionally, the sEH-related disease is hypertension (e.g., renal, hepatic, or pulmonary hypertension). Optionally, the sEH-related disease is adult respiratory distress syndrome. Optionally, the sEH-related disease is end stage renal disease. Optionally, the sEH-related disease is organ failure and/or damage (e.g., heart failure, renal failure, liver failure). Optionally, the sEH-related disease is cardiac fibrosis. Optionally, the sEH-related disease is renal fibrosis. Optionally, the sEH-related disease is peripheral vascular disease (e.g., ischemic limb disease, intermittent claudication, endothelial dysfunction, erectile dysfunction, Raynaud's disease, and diabetic vasculopathies). Optionally, the sEH-related disease is an atherothrombotic disorder (e.g., coronary artery disease, coronary vasospasm, angina, stroke, myocardial ischemia, myocardial infarction, hyperlipidemia). Optionally, the sEH-related disease is a metabolic disorder (e.g., diabetes).

The methods of treating or preventing a sEH-related disease (e.g., Alzheimer's disease) in a subject can further comprise administering to the subject a second compound, biomolecule, or compositions. The one or more additional agents and the compounds described herein or pharmaceutically acceptable salts or prodrugs thereof can be administered in any order, including concomitant, simultaneous, or sequential administration. Sequential administration can be administration in a temporally spaced order of up to several days apart. The methods can also include more than a single administration of the one or more additional agents and the compounds described herein or pharmaceutically acceptable salts or prodrugs thereof. The administration of the one or more additional agents and the compounds described herein or pharmaceutically acceptable salts or prodrugs thereof can be by the same or different routes and concurrently or sequentially.

Additional agents include, but are not limited to, anti-inflammatory agents. Examples of suitable anti-inflammatory agents include, for example, epoxyeicosatrienoic acid (EET), epoxydocosapentaenoic acid (EDP), and epoxyeicosaquatraeunoic acid (EEQ).

Any of the aforementioned therapeutic agents can be used in any combination with the compositions described herein. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

The methods and compounds as described herein are useful for both prophylactic and therapeutic treatment. For prophylactic use, a therapeutically effective amount of the compounds and compositions or pharmaceutically acceptable salts thereof as described herein are administered to a subject prior to onset (e.g., before obvious signs of a sEH-related disease), during early onset (e.g., upon initial signs and symptoms of a sEH-related disease), or after the development of a sEH related disease. Prophylactic administration can occur for several days to years prior to the manifestation of symptoms of a sEH-related disease. Therapeutic treatment involves administering to a subject a therapeutically effective amount of the compounds and compositions or pharmaceutically acceptable salts thereof as described herein after a sEH-related disease is diagnosed.

The methods of treating or preventing a sEH-related disease can also include administering the compounds or pharmaceutical compositions described herein by one or more clinically acceptable routes. The compounds or pharmaceutical compositions described herein can be administered orally, intraperitoneally, sublingually, subcutaneously, intravenously, or any clinically acceptable administration route. Optionally, the methods can further include selecting an elderly subject. Optionally, the methods can further include selecting a subject having an amyloid and/or tau biomarker.

The methods can also include administering the compounds or pharmaceutical compositions described herein that results in a certain brain to plasma ratio (B/P ratio) of the compound or compounds. The B/P ratio of the compound or compounds can be, but is not limited to, a ratio of least 0.3, a ratio from 0.1 to 0.99, a ratio from 0.1 to 0.5, a ratio from 0.3 to 0.4, a ratio from 0.3 to 0.5, a ratio from 0.3 to 0.6, a ratio from 0.3 to 0.7, a ratio from 0.3 to 0.8, a ratio from 0.3 to 0.9, a ratio from 0.5 to 0.6, a ratio from 0.5 to 0.7, a ratio from 0.5 to 0.8, a ratio from 0.5 to 0.9, a ratio from 0.5 to 0.95, a ratio from 0.6 to 0.7, a ratio from 0.6 to 0.8, a ratio from 0.6 to 0.9, a ratio from 0.6 to 0.95.

The compounds described herein are also useful in reducing amyloid beta plaque formation in a subject. The methods for reducing amyloid beta plaque formation in a subject include administering to the subject an effective amount of a compound or pharmaceutical composition described herein.

The compounds described herein are also useful in inhibiting soluble epoxide hydrolase in a cell. The methods for inhibiting soluble epoxide hydrolase in a cell include contacting a cell with an effective amount of one or more of the compounds as described herein. Optionally, the contacting is performed in vivo. Optionally, the contacting is performed in vitro.

V. Kits

Also provided herein are kits for treating or preventing a sEH-related disease (e.g., Alzheimer's disease, a neurodegenerative disorder, an inflammatory disease, and/or a metabolic disorder) in a subject. A kit can include any of the compounds or compositions described herein. For example, a kit can include one or more compounds of Formula I. A kit can further include one or more additional agents, such as one or more anti-inflammatory agents. A kit can include an oral formulation of any of the compounds or compositions described herein. A kit can include an intravenous formulation of any of the compounds or compositions described herein. A kit can additionally include directions for use of the kit (e.g., instructions for treating a subject), a container, a means for administering the compounds or compositions (e.g., a syringe), and/or a carrier.

As used herein the terms treatment, treat, or treating refer to a method of reducing one or more symptoms of a disease or condition. Thus in the disclosed method, treatment can refer to a 10%, 20%, 30%, 40%, 50%, 60%, 70%, 80%, 90%, or 100% reduction in the severity of one or more symptoms of the disease or condition. For example, a method for treating a disease is considered to be a treatment if there is a 1⁰% reduction in one or more symptoms or signs (e.g., cognitive function, brain amyloid and tau/NFT levels, hippocampal volume, and brain connectivity) of the disease in a subject as compared to a control. As used herein, control refers to the untreated condition (e.g., at-risk populations not treated with the compounds and compositions described herein). Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 1⁰%, 20%, 30%, 40%, 50%, 60%, 70%, 80%, 90% or greater as compared to a control level. Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs; and goats. Non-mammals include, for example, fish and birds.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Synthesis of Benimidazole Compounds

Benzimidazole compounds as described herein were synthesized according to the following general procedure: To a solution of benzene-1,2-diamine (1.1 mmol) and benzoic acid (1.0 mmol) in DMF (5 mL) were added EDCI (230 mg, 1.2 mmol) and HOBT (162 mg, 1.2 mmol), and the mixture was stirred for 4 hours followed by addition of HOAc (5 mL). The reaction was heated to 110° C. overnight and then concentrated under reduced pressure. The residue was extracted using ethyl acetate (3×10 mL), and the combined organic layers were washed with 1M NaOH and brine, respectively. The solvent was dried over NaSO₄ and removed under reduced pressure, and the residue was purified via column chromatography to give corresponding benzimidazole product. A general synthetic scheme is shown below:

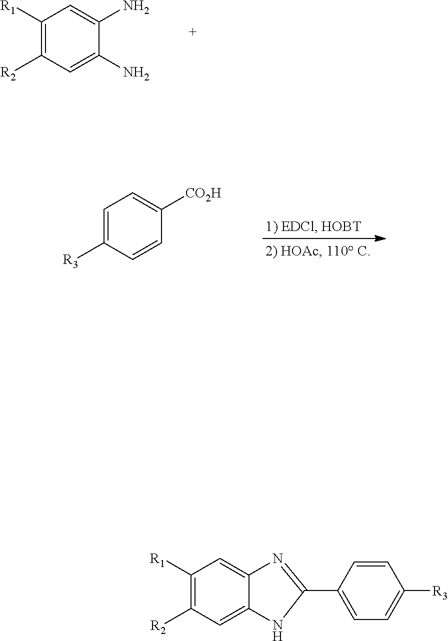

Characterization data for Compounds 1-61 are detailed below.

(S)-(5,6-difluoro-1λ²-benzo[d]imidazol-2-yl)(phenyl)methanamine (Compound 1): ¹H NMR (DMSO-d₆): δ=10.06 (s, 1H), 8.44 (d, 2H, J=4 Hz), 7.35 (m, 4H), 7.01 (d, 1H, J=4 Hz), 6.68 (d, 1H, J=4 Hz), 4.32 (m, 1H), 3.17 (dd, 2H, J=4 Hz, 20 Hz).

(S)-1-(5,6-difluoro-1λ²-benzo[d]imidazol-2-yl)ethan-1-amine (Compound 2): ¹H NMR (DMSO-d₆): δ=12.32 (s, 1H), 7.56 (m, 1H), 7.43 (m, 1H), 7.35 (m, 1H), 4.80 (m, 1H), 1.43 (d, 3H, J=7.2 Hz).

4-(5,6-dimethyl-1H-benzo[d]imidazol-2-yl)-N,N-dipropylbenzenesulfonamide (Compound 3): ¹H NMR (DMSO-d₆): δ=12.8 (bro, 1H), 8.29 (d, 2H, J=8.8 Hz), 7.90 (d, 2H, J=8.8 Hz), 7.39 (s, 2H), 3.05 (t, d, 4H, J=7.6 Hz), 2.30 (s, 6H), 1.47 (m, 4H), 0.79 (t, 6H, J=7.6 Hz).

5,6-dimethyl-N-(4-(trifluoromethoxy)phenyl)-1H-benzo[d]imidazol-2-amine (Compound 4): ¹H NMR (Methanol-d): δ=8.30 (bro. 1H), 7.52 (d, 2H, J=8.8 Hz), 7.29 (d, 2H, J=8.8 Hz), 7.12 (s, 2H), 6.81 (s, 1H), 2.31 (s, 6H).

2-(4-ethylphenyl)-5,6-dimethyl-1H-benzo[d]imidazole (Compound 5): ¹H NMR (DMSO-d₆): δ=13.53 (bro, 1H), 8.00 (d, 2H, J=8.0 Hz), 7.37 (s, 1H), 7.08 (d, 2H, =8.0 Hz), 7.24 (s, 1H), 2.63 (q, 2H, J=8.0 Hz), 2.29 (s, 3H), 2.28 (s, 3H), 1.19 (t, 3H, J=8.0 Hz).

N-(4-(tert-butyl)phenyl)-5,6-dimethyl-1H-benzo[d]imidazol-2-amine (Compound 6): ¹H NMR (CDCl₃): δ=9.6 (s, 1H), 7.08 (m, 4H), 7.00 (s, 2H), 2.21 (s, 6H), 1.20 (s, 9H).

N-(3,4-dimethoxyphenyl)-5,6-dimethyl-1H-benzo[d]imidazol-2-amine (Compound 7); ¹H NMR (CDCl₃): δ=8.43 (s, 1H), 7.05 (s, 2H), 6.82 (m, 1H), 6.74 (m, 2H), 6.24 (s, 1H), 3.75 (s, 3H), 3.74 (s, 3H), 2.25 (s, 6H).

2-(2-chlorophenyl)-1H-benzo[d]imidazole (Compound 8): ¹H NMR (DMSO-d₆): δ=13.0 (bro, 1H), 8.20 (s, 1H), 8.11 (m, 1H), 7.20 (m, 2H).

2-(5,6-dimethyl-1H-benzo[d]imidazol-2-yl)phenol (Compound 9): ¹H NMR (DMSO-d₆): δ=13.1 (bro, 1H), 7.97 (m, 1H), 7.40 (s, 2H), 7.32 (m, 1H), 6.97 (m, 2H), 2.31 (m, 6H).

2-(2,4-difluorophenyl)-5,6-dimethyl-1H-benzo[d]imidazole (Compound 10): ¹H NMR (DMSO-d₆): δ=12.3 (bro, 1H), 8.21 (m, 1H), 7.49 (m, 2H), 7.25 (m, 2H), 2.29 (s, 6H).

2-(4-bromophenoxy)-5,6-dimethyl-1H-benzo[d]imidazole (Compound 11): ¹H NMR (DMSO-d₆): δ=7.60 (d, 2H, J=8.8 Hz), 7.26 (m, 4H).

5,6-dimethyl-2-(4-(trifluoromethoxy)phenoxy)-1H-benzo[d]imidazole (Compound 12): ¹H NMR (Methanol-d₄): δ=7.31 (m, 4H), 7.13 (s, 2H), 2.30 (s, 6H).

N-(1H-benzo[d]imidazol-2-yl)-2-phenylacetamide (Compound 13): ¹H NMR (DMSO-d₆): δ=11.72 (bro, 1H), 7.41-7.21 (m, 8H), 7.05 (dd, 2H, J=2.8 Hz, 6.4 Hz), 3.75 (s, 2H).

1-(1H-benzo[d]imidazol-2-yl)-3-(3,5-bis(trifluoromethyl)phenyl)urea (Compound 14): ¹H NMR (DMSO-d₆): δ=11.79 (bro, 1H), 9.87 (s, 1H), 8.36 (s, 2H), 7.51 (s, 1H), 7.29 (dd, 2H, J=3.2 Hz, 5.6 Hz), 7.08 (dd, 2H, J=3.2 Hz, 6.0 Hz).

1,2-bis(1H-benzo[d]imidazol-2-yl)benzene (Compound 15): ¹H NMR (DMSO-d₆): δ=13.23 (s, 2H), 8.05 (m, 4H), 7.68 (m, 2H), 7.18 (m, 6H).

5,6-difluoro-2-(4-(trifluoromethyl)phenyl)-1H-benzo[d]imidazole (Compound 16): ¹H NMR (DMSO-d₆): δ=13.4 (bro, 1H), 8.33 (d, 2H, J=8.4 Hz), 7.91 (d, 2H, J=8.4 Hz), 7.68 (s, 2H).

2-([1,1′-biphenyl]-4-y)-5,6-difluoro-1H-benzo[d]imidazole (Compound 17): ¹H NMR (DMSO-d₆): δ=13.17 (bro, 1H), 8.21 (d, 2H, J=8.0 Hz), 7.85 (d, 2H, J=8.0 Hz), 7.75 (d, 2H, J=8.0 Hz), 7.74 (m, 1H), 7.51 (m, 1H), 7.51-7.46 (m, 2H), 7.41-7.32 (m, 1H).

5,6-dimethyl-2-(1-methylpiperidin-4-yl)-1H-benzo[d]imidazole (Compound 18): ¹H NMR (DMSO-d₆): δ=8.16 (s, 1H), 7.19 (s, 2H), 2.86-2.80 (m, 2H), 2.78-2.65 (m, 1H), 2.24 (s, 6H), 2.20 (s, 3H), 2.15-2.01 (m, 2H), 1.97-1.91 (m, 2H), 1.82-1.71 (m, 2H).

5,6-dimethyl-2-(4-(trifluoromethyl)phenyl)-1H-benzo[d]imidazole (Compound 19): ¹H NMR (DMSO-d₆): δ=8.12 (d, 1H, J=8.0 Hz), 7.84 (d, 1H, J=8.4 Hz), 7.41 (s, 1H), 7.38 (s, 1H), 2.30 (s, 3H), 2.22 (s, 3H).

2-([1,1′-biphenyl]-4-yl)-5,6-dimethyl-1H-benzo[d]imidazole (Compound 20): ¹H NMR (DMSO-d₆): δ=12.65 (bro, 1H), 8.19 (d, 1H, J=8.4 Hz), 7.82 (d, 1H, J=8.4 Hz), 7.73 (d, 1H, J=7.2 Hz), 7.49-7.27 (m, 5H), 2.31 (s, 3H), 2.29 (s, 3H).

5,6-dimethyl-2-(6-methylpyridin-3-yl)-1H-benzo[d]imidazole (Compound 21): ¹H NMR (DMSO-d₆): δ=12.72 (bro, 1H), 9.13 (d, 1H, J=2.0 Hz), 8.30 (dd, 1H, J=2.0 Hz, 8.0 Hz), 7.38 (d, 1H, J=8.4 Hz), 7.26 (s, 1H), 2.50 (s, 3H), 2.29 (s, 6H).

2-(5-fluoropyridin-2-yl)-5,6-dimethyl-1H-benzo[d]imidazole (Compound 22): ¹H NMR (DMSO-d₆): δ=12.90 (bro, 1H), 8.67 (d, 1H, J=4.8 Hz), 8.30 (dd, 1H, J=5.2 Hz, 8.8 Hz), 7.88 (m, 1H), 7.42 (s, 1H), 7.26 (s, 1H), 2.29 (s, 6H).

5-(5,6-dimethyl-1H-benzo[d]imidazol-2-yl)pyridin-2(1H)-one (Compound 23): ¹H NMR (DMSO-d₆): δ=12.38 (bro, 1H), 11.92 (bro, 1H), 8.11 (dd, 1H, J=2.8 Hz, 8.0 Hz), 7.26 (s, 2H), 6.44 (d, 1H, J=9.1 Hz), 2.47 (s, 3H), 2.27 (s, 6H).

2-(2-methoxypyridin-4-yl)-5,6-dimethyl-1H-benzo[d]imidazole (Compound 24): ¹H NMR (DMSO-d₆): δ=12.93 (bro, 1H), 8.27 (d, 1H, J=5.2 Hz), 7.64 (d, 1H, J=5.2 Hz), 7.45 (s, 2H), 7.30 (s, 1H), 3.89 (s, 3H), 2.30 (s, 6H).

5,6-dimethyl-2-(pyridin-2-yl)-1H-benzo[d]imidazole (Compound 25): ¹H NMR (DMSO-d₆): δ=12.81 (bro, 1H), 8.67 (d, 1H, J=4.8 Hz), 8.24 (d, 1H, J=7.6 Hz), 7.96-7.91 (m, 1H), 7.47 (m, 1H), 7.42 (s, 1H), 7.27 (s, 1H), 2.29 (s, 6H).

2-(4-(tert-butyl)phenyl)-5,6-dimethyl-1H-benzo[d]imidazole (Compound 26): ¹H NMR (DMSO-d₆): δ=8.12 (s, 1H), 8.02 (d, 2H, J=8.8 Hz), 7.51 (d, 2H, J=8.8 Hz), 7.37 (bro, 2H), 2.29 (s, 6H), 1.30 (s, 9H).

2-(3-(tert-butyl)phenyl)-5,6-dimethyl-1H-benzo[d]imidazole (Compound 27): ¹H NMR (DMSO-d₆): δ=12.59 (bro, 1H), 8.15 (s, 1H), 7.91 (d, 1H, J=7.8 Hz), 7.47-7.20 (M, 4H), 2.29 (s, 6H), 1.33 (s, 9H).

5,6-dimethyl-2-(naphthalen-2-yl)-1H-benzo[d]imidazole (Compound 28): ¹H NMR (DMSO-d₆): δ=10.17 (bro, 1H), 8.24-8.20 (m, 2H), 8.11-7.95 (m, 4H), 7.70-7.65 (m, 1H), 7.60-7.56 (m, 2H), 2.40 (s, 3H), 2.26 (s, 3H).

5,6-dimethyl-2-(pyridin-4-yl)-1H-benzo[d]imidazole (Compound 29): ¹H NMR (DMSO-d₆): δ=8.70 (d, 2H, J=6.0 Hz), 8.02 (d, 2H, J=6.0 Hz), 7.40 (s, 2H), 2.31 (s, 6H).

5,6-dimethyl-2-(pyridin-3-yl)-1H-benzo[d]imidazole (Compound 30): ¹H NMR (DMSO-d₆): δ=12.80 (bro, 1H), 9.27 (d, 2H, J=2.0 Hz), 8.61 (d, 2H, J=6.0 Hz), 8.41 (d, 2H, J=8.0 Hz), 7.52 (m, 1H), 7.40 (s, 1H), 7.29 (s, 1H), 2.30 (s, 6H).

5,6-dimethyl-2-(1-methyl-1H-pyrazol-5-yl)-1H-benzo[d]imidazole (Compound 31): ¹H NMR (DMSO-d₆): δ=12.62 (bro, 1H), 7.52 (d, 2H, J=6.0 Hz), 7.45 (s, 1H), 7.27 (s, 1H), 6.91 (d, 2H, J=6.0 Hz), 4.27 (s, 3H), 2.31 (s, 3H), 2.30 (s, 3H).

5,6-difluoro-2-(furan-3-yl)-1H-benzo[d]imidazole (Compound 32): ¹H NMR (DMSO-d₆): δ=12.95 (bro, 1H), 8.36 (s, 1H), 7.83 (s, 1H), 7.58 (bro. 2H), 7.03 (s, 1H).

4-(5,6-difluoro-1H-benzo[d]imidazol-2-yl)phenol (Compound 33): ¹H NMR (DMSO-d₆): δ=10.04 (bro, 1H), 8.11 (s, 1H), 7.94 (d, 2H, J=7.6 Hz), 8.35 (m, 2H), 6.90 (d, 2H, J=8.0 Hz).

5,6-dichloro-2-(4-(trifluoromethyl)phenyl)-1H-benzo[d]imidazole (Compound 34). ¹H NMR (DMSO-d₆): δ=8.35 (d, 2H, J=8.4 Hz), 7.93 (d, 2H, J=7.2 Hz), 7.89 (s, 2H).

2-(4-ethylphenyl)-5,6-difluoro-1H-benzo[d]imidazole imidazole (Compound 35): ¹H NMR (DMSO-d₆): δ=13.07 (bro, 1H), 8.03 (d, 2H, J=7.2 Hz), 7.58-7.40 (m, 2H), 7.36 (d, 2H, J=7.2 Hz), 2.66 (q, 2H, J=7.2 Hz), 1.20 (t, 3H, J=8.8 Hz).

2-(4-(trifluoromethyl)phenyl)-1H-benzo[d]imidazole (Compound 36): ¹H NMR (DMSO-d₆): δ=13.15 (bro, 1H), 8.36 (d, 2H, J=8.0 Hz), 7.91 (d, 2H, J=8.0 Hz), 7.68 (d, 2H, J=7.6 Hz), 7.54 (d, 2H, J=7.6 Hz), 7.25-7.20 (m, 2H).

2-(4-ethylphenyl)-1H-benzo[d]imidazole (Compound 37): ¹H NMR (DMSO-d₆): δ=12.78 (bro, 1H), 8.06 (d, 2H, J=8.0 Hz), 7.55 (m, 2H), 7.36 (d, 2H, J=8.0 Hz), 7.16 (s, 2H), 2.67 (q, 2H, J=7.6 Hz), 1.20 (t, 3H, J=7.6 Hz).

5,6-dibromo-2-(4-(trifluoromethyl)phenyl)-1H-benzo[d]imidazole (Compound 38): ¹H NMR (DMSO-d₆): δ=8.35 (d, 2H, J=8.0 Hz), 8.02 (s, 2H), 7.93 (d, 2H, J=8.0 Hz).

5,6-dibromo-2-(4-ethylphenyl)-1H-benzo[d]imidazole (Compound 39): ¹H NMR (DMSO-d₆): δ=8.05 (d, 2H, J=7.6 Hz), 7.94 (s, 2H), 7.38 (d, 2H, J=7.6 Hz), 2.65 (q, 2H, J=7.6 Hz), 8.05 (t, 3H, J=7.6 Hz).

5-(tert-butyl)-2-(4-(trifluoromethyl)phenyl)-1H-benzo[d]imidazole (Compound 40): ¹H NMR (DMSO-d₆): δ=13.00 (bro, 1H), 8.32 (d, 2H, J=8.0 Hz), 7.90 (d, 2H, J=7.6 Hz), 7.59 (m, 1H), 7.46 (s, 1H), 7.32 (s, 1H), 1.34 (s, 9H).

4,5-dimethyl-2-(4-(trifluoromethyl)phenyl)-1H-benzo[d]imidazole (Compound 41): ¹H NMR (DMSO-d₆): δ=12.59 (bro, 1H), 8.41-8.34 (m, 2H), 8.00-7.83 (m, 2H), 7.38 (m, 1H), 7.03 (m, 1H), 2.32 (s, 3H), 2.28 (m, 3H).

5,6-dimethoxy-2-(4-(trifluoromethyl)phenyl)-1H-benzo[d]imidazole (Compound 42): ¹H NMR (DMSO-d₆): δ=8.26 (d, 2H, J=7.6 Hz), 7.86 (d, 2H, J=7.6 Hz), 7.22 (s, 1H), 7.00 (s, 1H), 3.79 (s, 6H).

6-chloro-5-fluoro-2-(4-(trifluoromethyl)phenyl)-1H-benzo[d]imidazole (Compound 43): ¹H NMR (DMSO-d₆): δ=8.34 (d, 2H, J=8.0 Hz), 7.92 (d, 2H, J=7.6 Hz), 7.82 (d, 1H, J=7.2 Hz), 7.66 (d, 1K J=9.6 Hz).

6-bromo-5-fluoro-2-(4-(trifluoromethyl)phenyl)-1H-benzo[d]imidazole (Compound 44): ¹H NMR (DMSO-d₆): δ=13.40 (bro, 1H), 8.34 (d, 2H, J=8.0 Hz), 7.92 (d, 2H, J=8.4 Hz), 7.91 (s, 1H), 7.65 (s, 1H).

2-([1,1′-biphenyl]-4-yl)-6-bromo-5-fluoro-1H-benzo[d]imidazole (Compound 45); ¹H NMR (DMSO-d₆): δ=13.22 (bro, 1H), 8.22 (d, 2H, J=8.4 Hz), 7.86 (d, 2H, J=8.0 Hz), 7.83 (s, 1H), 7.76 (d, 2H, J=8.0 Hz), 7.62 (s, 1H), 7.48-7.42 (m, 2H), 7.39 (m, 1H).

5,6-difluoro-2-(4-methoxyphenyl)-1H-benzo[d]imidazole (Compound 46): ¹H NMR (DMSO-d₆): δ=13.00 (bro, 1H), 8.04 (d, 2H, J=8.4 Hz), 7.56 (bro, 2 Hz), 7.08 (d, 2H, J=8.4 Hz), 3.81 (s, 3H).

N-(4-(5,6-difluoro-1H-benzo[d]imidazol-2-yl)phenyl)acetamide (Compound 47): ¹H NMR (DMSO-d₆): δ=13.00 (bro, 1H), 10.16 (s, 1H), 8.03 (d, 2H, J=8.4 Hz), 7.71 (d, 2H, J=8.4 Hz), 7.64 (s, 1H), 7.52 (s, 1H), 2.06 (s, 3H).

4,5-difluoro-2-(4-(trifluoromethyl)phenyl)-1H-benzo[d]imidazole (Compound 48): ¹H NMR (DMSO-d₆): δ=8.36 (d, 2H, J=8.4 Hz), 7.92 (d, 2H, J=8.4 Hz), 7.41 (m, 1H), 7.36-7.24 (m, 1H).

2-([1,1′-biphenyl]-4-yl)-4,5-difluoro-1H-benzo[d]imidazole (Compound 49): ¹H NMR (DMSO-d₆): δ=13.36 (bro, 1H), 8.25 (d, 2H, J=7.6 Hz), 7.87 (d, 2H, J=8.0 Hz), 7.76 (d, 2H, J=7.6 Hz), 7.62 (m, 2H), 7.51-7.45 (m, 2H), 7.42-7.19 (m, 1H).

5,6-difluoro-2-(2-(trifluoromethyl)pyridin-4-yl)-1H-benzo[d]imidazole (Compound 50): ¹H NMR (DMSO-d₆): δ=8.92 (d, 2H, J=4.4 Hz), 8.53 (s, 1H), 8.35 (d, 2H, J=4.4 Hz), 7.79 (m, 2H).

2-(4-(5,6-difluoro-1H-benzo[d]imidazol-2-yl)phenyl)thiazole (Compound 51): ¹H NMR (DMSO-d₆): δ=8.47 (d, 1H, J=8.4 Hz), 8.39 (d, 2H, 1=8.4 Hz), 7.77 (m, 2H), 7.67 (d, 1H, J=5.2 Hz), 7.43 (d, 1H, J=5.2 Hz).

5,6-difluoro-2-(4-(4-fluorophenoxy)phenyl)-1H-benzo[d]imidazole (Compound 52): ¹H NMR (CDCl₃): δ=7.93 (d, 2H, J=8.8 Hz), 7.38 (m, 2H), 7.09-7.04 (m, 6H).

5,6-difluoro-2-(4-phenoxyphenyl)-1H-benzo[d]imidazole (Compound 53): ¹H NMR (DMSO-d₆): δ=13.23 (bro, 1H), 8.10 (d, 2H, J=8.4 Hz), 7.63-7.51 (m, 2H), 7.44-7.40 (m, 2H), 7.17 (m, 1H), 7.12-7.07 (m, 5H).

4-(5,6-difluoro-1H-benzo[d]imidazol-2-yl)benzonitrile (Compound 54): ¹H NMR (DMSO-d₆): δ=8.26 (d, 2H, J=8.8 Hz), 8.00 (d, 2H, J=8.8 Hz), 7.64 (m, 2H).

5-(4-(5,6-difluoro-1H-benzo[d]imidazol-2-yl)phenyl)-1,3,4-oxadiazol-2(3H)-one (Compound 55): ¹H NMR (DMSO-d₆): δ=8.24 (d, 2H, J=8.4 Hz), 7.92 (d, 2H, J=8.4 Hz), 7.64 (m, 2H).

5,6-difluoro-2-(4-phenoxyphenyl)-1H-benzo[d]imidazole (Compound 56): ¹H NMR (DMSO-d₆): δ=13.39 (bro, 1H), 8.21 (d, 2H, J=8.4 Hz), 8.06 (d, 2H, J=8.4 Hz), 7.70-7.60 (m, 2H).

Methyl 4-(5,6-difluoro-1H-benzo[d]imidazol-2-yl)benzoate (Compound 57): ¹H NMR (DMSO-d₆): δ=8.24 (d, 2H, J=8.0 Hz), 8.09 (d, 2H, J=8.0 Hz), 7.62 (m, 2H), 3.86 (s, 3H).

2-(4-(5,6-difluoro-1H-benzo[d]imidazol-2-yl)phenyl)-1,3,4-oxadiazole (Compound 58): ¹H NMR (DMSO-d₆): δ=9.36 (s, 1H), 8.32 (d, 2H, J=8.4 Hz), 8.17 (d, 2H, J=8.4 Hz), 7.67 (t, 2H, J=9.2 Hz).

5,6-difluoro-2-(4-(pyridin-4-yl)phenyl)-1H-benzo[d]imidazole (Compound 59): ¹H NMR (DMSO-d₆): δ=13.30 (bro, 1H), 8.64 (s, 2H), 8.25 (d, 2H, J=8.0 Hz), 8.08-7.94 (m, 3H), 7.79-7.71 (m, 2H).

5-(4-(5,6-difluoro-1H-benzo[d]imidazol-2-yl)phenyl)-3-methyl-1,2,4-oxadiazole (Compound 60): ¹H NMR (DMSO-d₆): δ=8.33 (d, 2H, J=8.4 Hz), 8.22 (d, 2H, J=8.4 Hz), 7.67 (m, 2H).

2-(4-(1H-tetrazol-5-yl)phenyl)-5,6-difluoro-1H-benzo[d]imidazole (Compound 61): ¹H NMR (DMSO-d₆): δ=8.31 (d, 2H, J=8.8 Hz), 8.18 (d, 2H, J=8.8 Hz), 7.64 (bro, 2H).

### Example 2: Structure-Activity Relationship of Benzimidazole-Based Compounds

The benzimidazole-based compounds synthesized according to Example 1 were measured for activity. Using a fluorescence-based sEH biochemical inhibition assay, the IC₅₀ value was measured for each benzimidazole-based compound. The results are summarized in Table 1. Human sEH (hsEH) and its substrate PHOME were purchased from Cayman Chemicals. IC₅₀ values of hsEH inhibitors were determined by fluorescence. IC₅₀ is defined as the concentration of the inhibitor that blocks 50% of the enzymatic activity. To a 96-well plate containing 10 μL enzyme (5 nM in water), 60 μL of H₂O and 10 μL BisTris-HCl buffer (25 mM, pH 7.0) were added inhibitors with different concentrations. The mixture was incubated for 5 min at 30° C. prior to addition of 10 μL of PHOME substrate (50 μM). Fluorescence were measured at 30° C. with an excitation wavelength of 330 nm and an emission wavelength of 465 nm using a BioTek H1 plate reader. The IC₅₀ values were calculated by non-linear regression fitting. The curve was generated from at least three separate runs, each in duplicates. Results are given as means and standard deviations. In Table 1, an IC₅₀ value less than or equal to 1 μM is indicated as an “A.” an IC₅₀ value from greater than 1 μM to less than 10 μM is indicated as a “B′” and an IC₅₀ value of greater than or equal to 10 μM is indicated as a “C.”

TABLE 1

| Compound Name | Structure | IC₅₀ |
|---|---|---|
| Compound 1 | 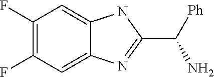 [SMILES: N[C@@H](c1ccccc1)c1nc2cc(F)c(F)cc2[nH]1] | C |
| Compound 2 | 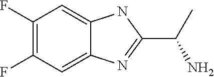 | C |
| Compound 3 | 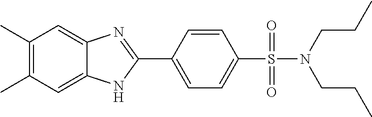 [SMILES: CCCN(CCC)S(=O)(=O)c1ccc(-c2nc3cc(C)c(C)cc3[nH]2)cc1] | B |
| Compound 4 | 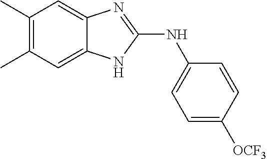 [SMILES: Cc1cc2nc(Nc3ccc(OC(F)(F)F)cc3)[nH]c2cc1C] | B |
| Compound 5 | 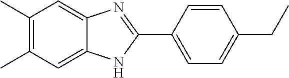 [SMILES: CCc1ccc(-c2nc3cc(C)c(C)cc3[nH]2)cc1] | A |
| Compound 6 | 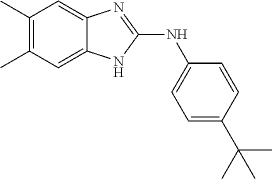 [SMILES: Cc1cc2nc(Nc3ccc(C(C)(C)C)cc3)[nH]c2cc1C] | B |
| Compound 7 | 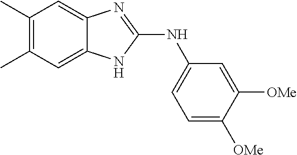 [SMILES: COc1ccc(Nc2nc3cc(C)c(C)cc3[nH]2)cc1OC] | B |
| Compound 8 | 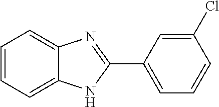 [SMILES: Clc1cccc(-c2nc3ccccc3[nH]2)c1] | C |
| Compound 9 | 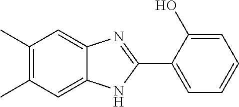 [SMILES: Cc1cc2nc(-c3ccccc3O)[nH]c2cc1C] | C |
| Compound 10 | 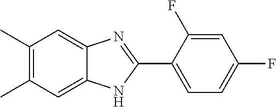 [SMILES: Cc1cc2nc(-c3ccc(F)cc3F)[nH]c2cc1C] | C |
| Compound 11 | 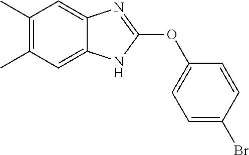 [SMILES: Cc1cc2nc(Oc3ccc(Br)cc3)[nH]c2cc1C] | C |
| Compound 12 | 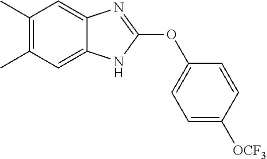 [SMILES: Cc1cc2nc(Oc3ccc(OC(F)(F)F)cc3)[nH]c2cc1C] | C |
| Compound 13 | 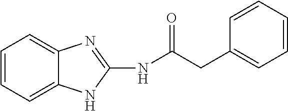 [SMILES: O=C(Cc1ccccc1)Nc1nc2ccccc2[nH]1] | C |
| Compound 14 | 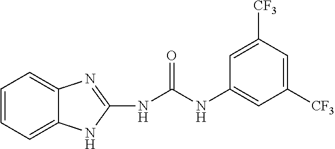 [SMILES: O=C(Nc1cc(C(F)(F)F)cc(C(F)(F)F)c1)Nc1nc2ccccc2[nH]1] | C |
| Compound 15 | 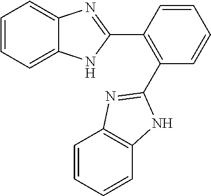 [SMILES: c1ccc(-c2nc3ccccc3[nH]2)c(-c2nc3ccccc3[nH]2)c1] | C |
| Compound 16 | 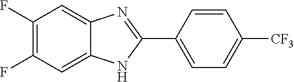 [SMILES: Fc1cc2nc(-c3ccc(C(F)(F)F)cc3)[nH]c2cc1F] | A |
| Compound 17 | 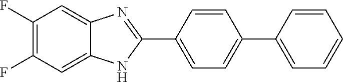 [SMILES: Fc1cc2nc(-c3ccc(-c4ccccc4)cc3)[nH]c2cc1F] | B |
| Compound 18 | 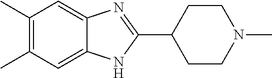 | C |
| Compound 19 | 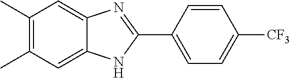 [SMILES: Cc1cc2nc(-c3ccc(C(F)(F)F)cc3)[nH]c2cc1C] | A |
| Compound 20 | 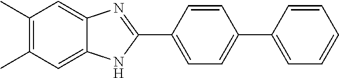 [SMILES: Cc1cc2nc(-c3ccc(-c4ccccc4)cc3)[nH]c2cc1C] | A |
| Compound 21 | 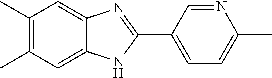 | B |
| Compound 22 | 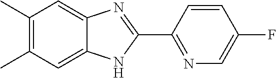 [SMILES: Cc1cc2nc(-c3ccc(F)cn3)[nH]c2cc1C] | B |
| Compound 23 | 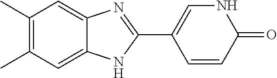 [SMILES: Cc1cc2nc(-c3ccc(=O)[nH]c3)[nH]c2cc1C] | C |
| Compound 24 | 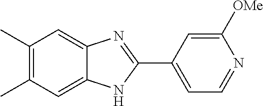 [SMILES: COc1cc(-c2nc3cc(C)c(C)cc3[nH]2)ccn1] | B |
| Compound 25 | 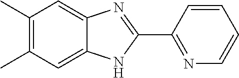 [SMILES: Cc1cc2nc(-c3ccccn3)[nH]c2cc1C] | C |
| Compound 26 | 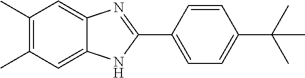 [SMILES: Cc1cc2nc(-c3ccc(C(C)(C)C)cc3)[nH]c2cc1C] | A |
| Compound 27 | 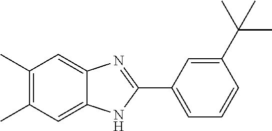 [SMILES: Cc1cc2nc(-c3cccc(C(C)(C)C)c3)[nH]c2cc1C] | A |
| Compound 28 | 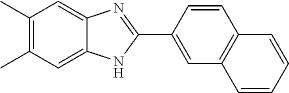 [SMILES: Cc1cc2nc(-c3ccc4ccccc4c3)[nH]c2cc1C] | A |
| Compound 29 | 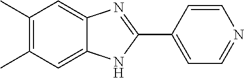 [SMILES: Cc1cc2nc(-c3ccncc3)[nH]c2cc1C] | C |
| Compound 30 | 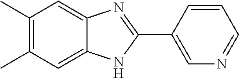 [SMILES: Cc1cc2nc(-c3cccnc3)[nH]c2cc1C] | C |
| Compound 31 | 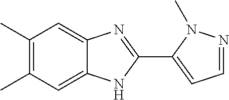 [SMILES: Cc1cc2nc(-c3ccnn3C)[nH]c2cc1C] | C |
| Compound 32 | 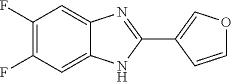 [SMILES: Fc1cc2nc(-c3ccoc3)[nH]c2cc1F] | C |
| Compound 33 | 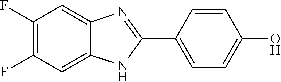 [SMILES: Oc1ccc(-c2nc3cc(F)c(F)cc3[nH]2)cc1] | C |
| Compound 34 | 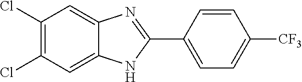 [SMILES: FC(F)(F)c1ccc(-c2nc3cc(Cl)c(Cl)cc3[nH]2)cc1] | B |
| Compound 35 | 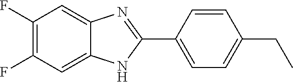 [SMILES: CCc1ccc(-c2nc3cc(F)c(F)cc3[nH]2)cc1] | A |
| Compound 36 | 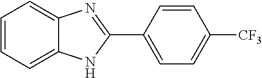 [SMILES: FC(F)(F)c1ccc(-c2nc3ccccc3[nH]2)cc1] | B |
| Compound 37 | 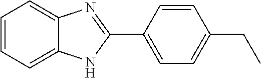 [SMILES: CCc1ccc(-c2nc3ccccc3[nH]2)cc1] | B |
| Compound 38 | 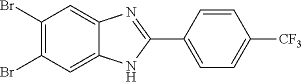 [SMILES: FC(F)(F)c1ccc(-c2nc3cc(Br)c(Br)cc3[nH]2)cc1] | C |
| Compound 39 | 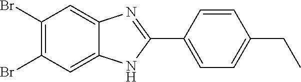 [SMILES: CCc1ccc(-c2nc3cc(Br)c(Br)cc3[nH]2)cc1] | B |
| Compound 40 | 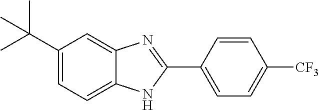 [SMILES: CC(C)(C)c1ccc2[nH]c(-c3ccc(C(F)(F)F)cc3)nc2c1] | B |
| Compound 41 | 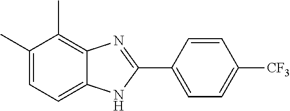 [SMILES: Cc1ccc2[nH]c(-c3ccc(C(F)(F)F)cc3)nc2c1C] | A |
| Compound 42 | 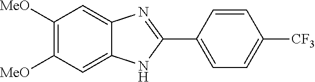 [SMILES: COc1cc2nc(-c3ccc(C(F)(F)F)cc3)[nH]c2cc1OC] | B |
| Compound 43 | 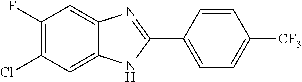 [SMILES: Fc1cc2nc(-c3ccc(C(F)(F)F)cc3)[nH]c2cc1Cl] | A |
| Compound 44 | 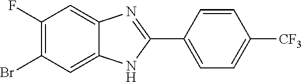 [SMILES: Fc1cc2nc(-c3ccc(C(F)(F)F)cc3)[nH]c2cc1Br] | A |
| Compound 45 | 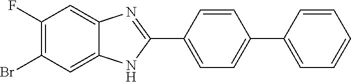 [SMILES: Fc1cc2nc(-c3ccc(-c4ccccc4)cc3)[nH]c2cc1Br] | A |
| Compound 46 | 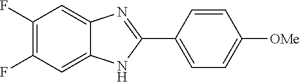 [SMILES: COc1ccc(-c2nc3cc(F)c(F)cc3[nH]2)cc1] | A |
| Compound 47 | 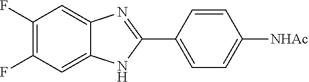 [SMILES: CC(=O)Nc1ccc(-c2nc3cc(F)c(F)cc3[nH]2)cc1] | C |
| Compound 48 | 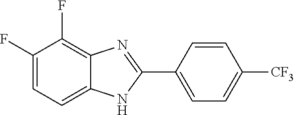 [SMILES: Fc1ccc2[nH]c(-c3ccc(C(F)(F)F)cc3)nc2c1F] | A |
| Compound 49 | 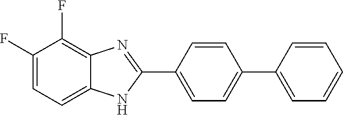 [SMILES: Fc1ccc2[nH]c(-c3ccc(-c4ccccc4)cc3)nc2c1F] | C |
| Compound 50 | 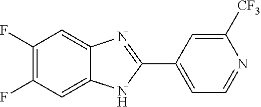 [SMILES: Fc1cc2nc(-c3ccnc(C(F)(F)F)c3)[nH]c2cc1F] | B |
| Compound 51 | 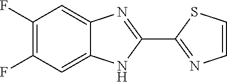 [SMILES: Fc1cc2nc(-c3nccs3)[nH]c2cc1F] | C |
| Compound 52 | 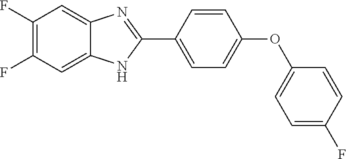 [SMILES: Fc1ccc(Oc2ccc(-c3nc4cc(F)c(F)cc4[nH]3)cc2)cc1] | A |
| Compound 53 | 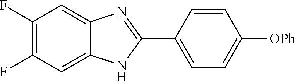 [SMILES: Fc1cc2nc(-c3ccc(Oc4ccccc4)cc3)[nH]c2cc1F] | A |
| Compound 54 | 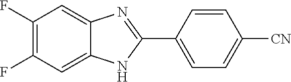 [SMILES: N#Cc1ccc(-c2nc3cc(F)c(F)cc3[nH]2)cc1] | A |
| Compound 55 | 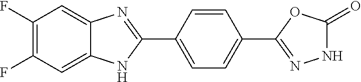 [SMILES: O=c1[nH]nc(-c2ccc(-c3nc4cc(F)c(F)cc4[nH]3)cc2)o1] | A |
| Compound 56 | 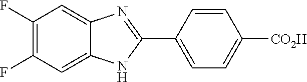 [SMILES: O=C(O)c1ccc(-c2nc3cc(F)c(F)cc3[nH]2)cc1] | A |
| Compound 57 | 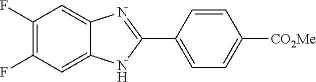 [SMILES: COC(=O)c1ccc(-c2nc3cc(F)c(F)cc3[nH]2)cc1] | C |
| Compound 58 | 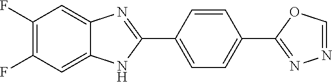 [SMILES: Fc1cc2nc(-c3ccc(-c4nnco4)cc3)[nH]c2cc1F] | C |
| Compound 59 | 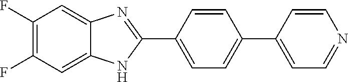 [SMILES: Fc1cc2nc(-c3ccc(-c4ccncc4)cc3)[nH]c2cc1F] | B |
| Compound 60 | 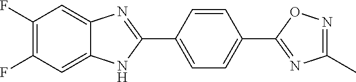 [SMILES: Cc1noc(-c2ccc(-c3nc4cc(F)c(F)cc4[nH]3)cc2)n1] | C |
| Compound 61 | 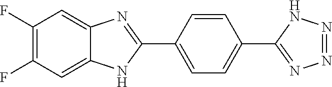 [SMILES: Fc1cc2nc(-c3ccc(-c4nnn[nH]4)cc3)[nH]c2cc1F] | C |

### Example 3: Activity Data for Additional Benzimidazole-Based Compounds

Additional benzimidazole-based compounds as described herein were measured for activity according to the procedure outlined in Example 2. Briefly, using a fluorescence-based sEH biochemical inhibition assay, the IC₅₀ value was measured for each benzimidazole-based compound. The tested compounds are shown in Table 2 and 3. All compounds in Table 2 exhibited an IC₅₀ value less than 100 nM. All compounds in Table 3 exhibited an IC₅₀ value less than 1 μM.

TABLE 2

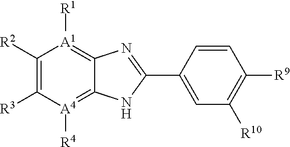

| Compound | A¹—R¹ | A⁴—R⁴ | R² | R³ | R⁹ | R¹⁰ |
|---|---|---|---|---|---|---|
| Compound | CH | CH | F | F | CF₃ | H |
| 16 | | | | | | |
| Compound 55 | CH | CH | F | F | 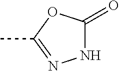 | H |
| Compound 62 | CH | CH | F | 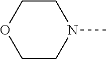 | CF₃ | H |
| Compound 63 | CH | CH | F | 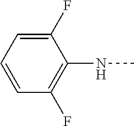 | CF₃ | H |
| Compound 64 | CH | CH | F | 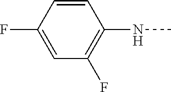 | CF₃ | H |
| Compound 65 | CH | CH | F | 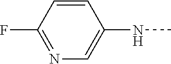 | CF₃ | H |
| Compound 66 | CH | CH | F | 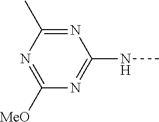 | CF₃ | H |
| Compound 67 | CH | CH | F | 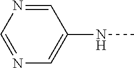 | CF₃ | H |
| Compound 68 | CH | CH | F | 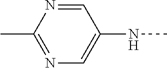 | CF₃ | H |
| Compound 69 | CH | CH | F | 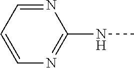 | CF₃ | H |
| Compound 70 | CH | CH | F | 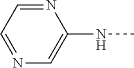 | CF₃ | H |
| Compound 71 | CH | CH | F | 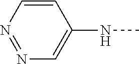 | CF₃ | H |
| Compound 72 | CH | CH | F | 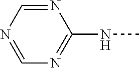 | CF₃ | H |
| Compound 73 | CH | CH | F | 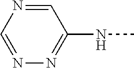 | CF₃ | H |
| Compound 74 | CH | CH | F | 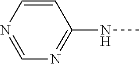 | CF₃ | H |
| Compound 75 | CH | CH | F | 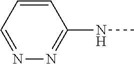 | CF₃ | H |
| Compound 76 | CH | CH | F | 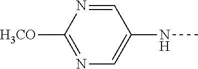 | CF₃ | H |
| Compound 77 | CH | CH | H | 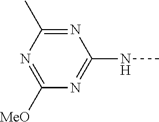 | CF₃ | H |
| Compound 78 | CH | CH | H | 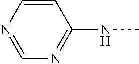 | CF₃ | H |
| Compound 79 | CH | CH | H | 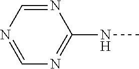 | CF₃ | H |
| Compound 80 | N (R¹ is absent) | N (R⁴ is absent) | H | 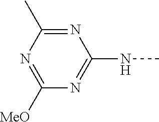 | CF₃ | H |
| Compound 81 | CH | CH | H | 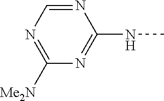 | CF₃ | H |
| Compound 82 | CH | CH | F | 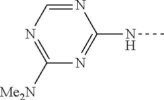 | CF₃ | H |
| Compound 83 | CH | CH | H | 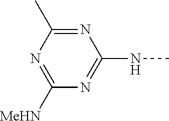 | CF₃ | H |
| Compound 84 | CH | CH | F | 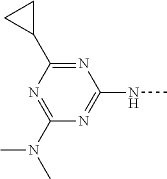 | CF₃ | H |
| Compound 85 | CH | CH | H | 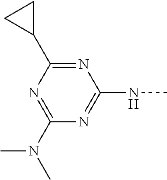 | CF₃ | H |
| Compound 86 | CH | CH | H | 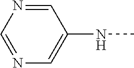 | CF₃ | H |
| | | | | X = CF | | |
| Compound 87 | CH | CH | H | 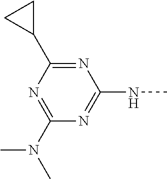 | CF₃ | H |
| | | | | X = CF | | |
| Compound 88 | CH | CH | F | 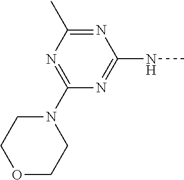 | CF₃ | H |
| Compound 89 | CH | CH | H | 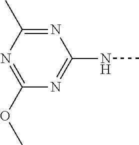 | CF₃ | H |
| | | | | X = CF | | |
| Compound 90 | CH | CH | F | 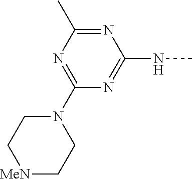 | CF₃ | H |
| Compound 91 | CH | CH | F | 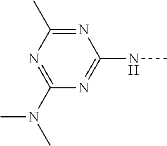 | CF₃ | H |
| Compound | CH | CH | F | F | F | CF₃ |
| 92 | | | | | | |
| Compound | CH | CH | F | F | CF₃ | F |
| 93 | | | | | | |
| Compound 94 | CH | CH | F | 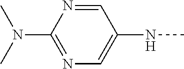 | CF₃ | H |
| Compound 95 | CH | CH | H | 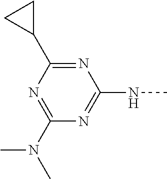 | CF₃ | H |
| | | | | X = CCl | | |
| Compound | CH | CH | F | F | CF(CF₃)₂ | H |
| 96 | | | | | | |
| Compound 97 | CH | CH | F | 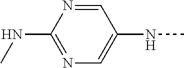 | CF₃ | H |
| Compound 98 | CH | CH | F | 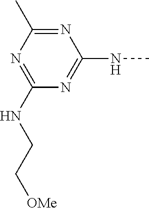 | CF₃ | H |
| Compound 99 | CH | CH | F | 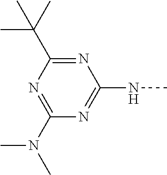 | CF₃ | H |
| Compound 100 | CH | CH | OMe | 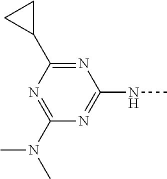 | CF₃ | F |
| Compound 101 | CH | CH | F | 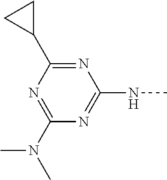 | CF₃ | F |
| Compound 102 | CH | CH | F | 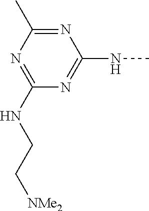 | CF₃ | H |

TABLE 3

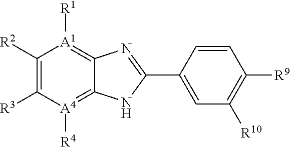

| Compound | A¹—R¹ | A⁴—R⁴ | R² | R³ | R⁹ | R¹⁰ |
|---|---|---|---|---|---|---|
| Compound 117 | CH | CH | F | F | CF₃ | 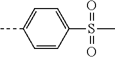 |
| Compound 118 | CH | CH | F | F | CF₃ | 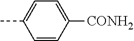 |
| Compound 119 | CH | CH | F | F | CF₃ | 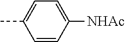 |
| Compound 120 | CH | CH | F | F | CF₃ | 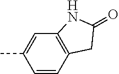 |
| Compound 121 | CH | CH | F | F | F | 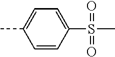 |
| Compound 122 | CH | CH | F | F | F | 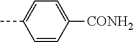 |
| Compound 123 | CH | CH | F | F | F | 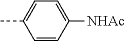 |
| Compound 124 | CH | CH | F | F | F | 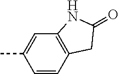 |
| Compound 125 | CH | CH | F | F | H | 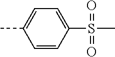 |
| Compound 126 | CH | CH | F | F | H | 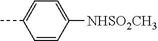 |
| Compound 127 | CH | CH | F | F | H | 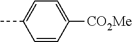 |
| Compound 128 | CH | CH | F | F | H | 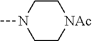 |

### Example 4: Cellular Activity Data

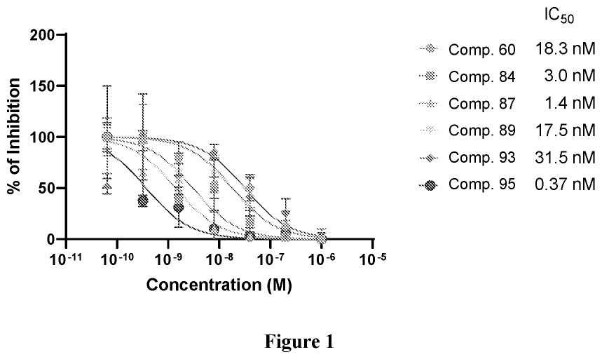

Benzimidazole-based compounds as described herein were measured for cellular sEH activity by measuring the conversion of 14,15-epoxyeicosatrienoic acid (14,15-EET) to 14,15-dihydroxyeicosatrienoic acid (14,15-DHET). HepG2 cells (20,000) in 100 μL of Opti-MEM were treated with compounds as described herein in doses ranging from 1.0 μM to 0.064 nM (7 doses of 5× serial dilutions) for 30 minutes incubation at 37° C. The tested compounds include Compound 66, Compound 84, Compound 87, Compound 89, Compound 93, and Compound 95. Then 14,15-EET (1.0 μM) was added to the cells and the cells were incubated an additional 2 hours. Twenty-five μL of non-diluted supernatant was subjected to LC-MS measurement to quantify the product 14,15-DHET. The data are shown in FIG. 1.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A compound of the following formula: 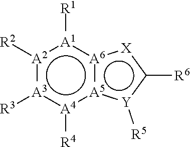
or a pharmaceutically acceptable salt or prodrug thereof, wherein:
A¹, A², A³, A⁴, A⁵, and A⁶ are each independently selected from the group consisting of N and C;
R¹, R², R³, and R⁴ are each independently selected from the group consisting of hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof,
R⁵ is hydrogen, deuterium, tritium, halogen, cyano, trifluoromethyl, alkoxy, cycloalkoxy, aryloxy, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl, or an isotopic substitution thereof,
R⁶ is hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl, or an isotopic substitution thereof;
X is selected from the group consisting from N and CR⁷, wherein R⁷ is hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, or substituted or unsubstituted heterocycloalkyl, or an isotopic substitution thereof; and
Y is N, O, or S,
wherein when A¹, A², A³, or A⁴ is N, then the directly bonded substituent selected from R¹, R², R³, or R⁴, respectively, is absent; and
wherein when Y is O or S, R⁵ is absent.

2. The compound of claim 1, wherein the compound has the following formula:

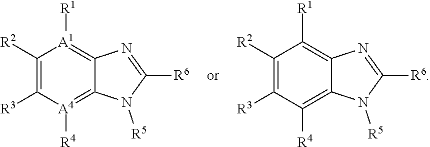

3. (canceled)

4. The compound of claim 1, wherein the compound has the following formula: 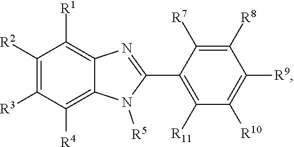
wherein
R⁷, R⁸, R⁹, R¹⁰, and R¹¹ are each independently selected from the group consisting of hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof.

5. The compound of claim 1, wherein the compound has the following formula: 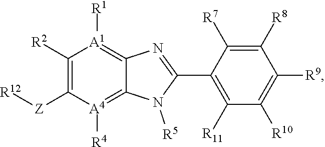
wherein
R⁷, R⁸, R⁹, R¹⁰, R¹¹, and R¹² are each independently selected from the group consisting of hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof; and
Z is CR¹³R¹⁴, NR¹³, O, S, or SO₂, wherein R¹³ and R¹⁴ are each independently selected from the group consisting of hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof.

6. The compound of claim 5, wherein R¹² or R¹³ is a substituted aryl or substituted heteroaryl.

7. The compound of claim 5, wherein R¹² or R¹³ is 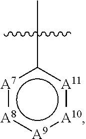
wherein
A⁷, A⁸, A⁹, A¹⁰, and A¹¹ are each independently selected from CR, N, N—OR, S, and O, and wherein each R is independently hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof.

8. The compound of claim 5, wherein R¹² or R¹³ is 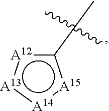
wherein
A¹², A¹³, A¹⁴, and A¹⁵ are each independently selected from CR, N, N—OR, S, and O, wherein each R is independently hydrogen, deuterium, tritium, halogen, cyano, trifluoromethyl, alkoxy, cycloalkoxy, aryloxy, substituted or unsubstituted amino, substituted or unsubstituted thio, substituted or unsubstituted sulfonyl, substituted or unsubstituted alkyl, substituted or unsubstituted alkenyl, substituted or unsubstituted alkynyl, substituted or unsubstituted heteroalkyl, substituted or unsubstituted heteroalkenyl, substituted or unsubstituted heteroalkynyl, substituted or unsubstituted aryl, substituted or unsubstituted heteroaryl, substituted or unsubstituted cycloalkyl, and substituted or unsubstituted heterocycloalkyl, or an isotopic substitution thereof.

9. The compound of claim 1, wherein the compound has the following formula selected from the group consisting of:

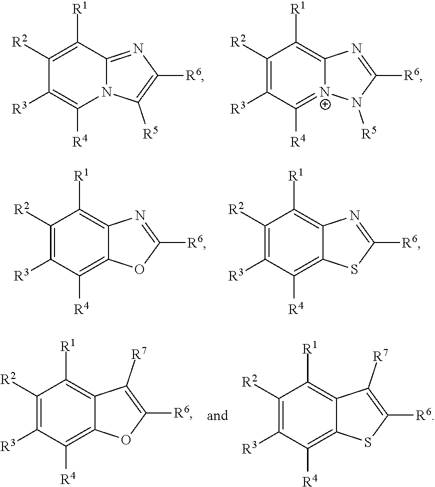

10. (canceled)

11. (canceled)

12. (canceled)

13. (canceled)

14. (canceled)

15. The compound of claim 1, wherein the compound is selected from the group consisting of:

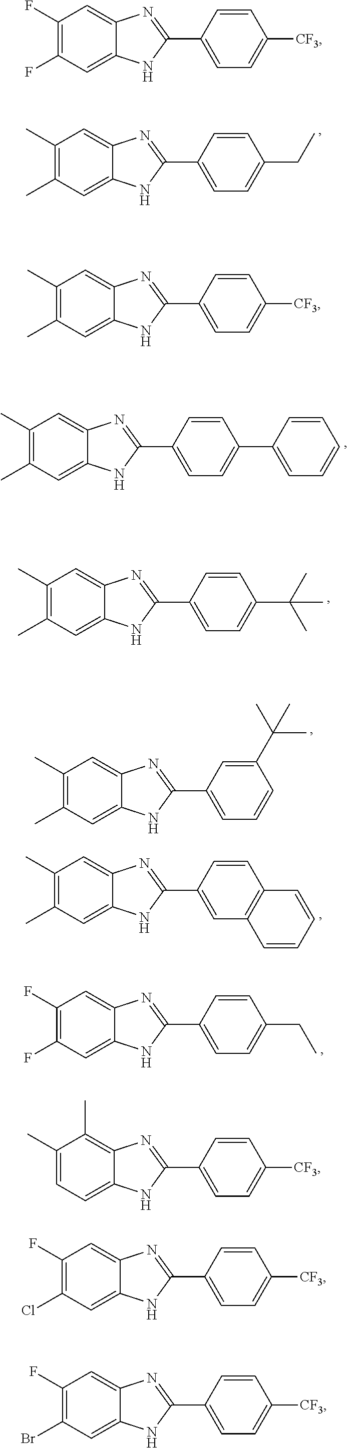

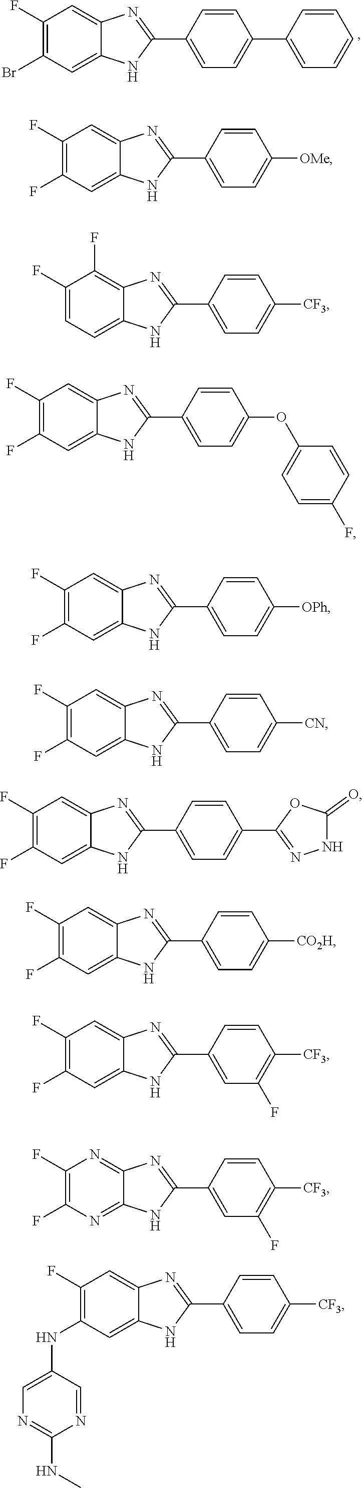

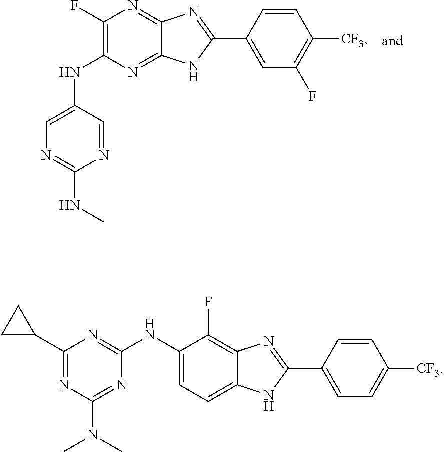 [SMILES: CNc1ncc(Nc2nc3[nH]c(-c4ccc(C(F)(F)F)c(F)c4)nc3nc2F)cn1 ; "and" ; CN(C)c1nc(Nc2ccc3[nH]c(-c4ccc(C(F)(F)F)cc4)nc3c2F)nc(C2CC2)n1]

16. A pharmaceutical composition comprising a compound of claim 1 and a pharmaceutically acceptable carrier.

17. (canceled)

18. A method of treating or preventing a soluble epoxide hydrolase-related disease in a subject, comprising:
administering to the subject an effective amount of a compound of claim 1.

19. The method of claim 18, wherein the soluble epoxide hydrolase-related disease is a neurodegenerative disorder.

20. The method of claim 19, wherein the neurodegenerative disorder is Alzheimer's disease, Parkinson's disease, dementia, cerebral ischemia, a seizure, traumatic brain injury, stroke, Alexander disease, Alper's disease, amyotrophic lateral sclerosis, ataxia telangiectasia, Batten disease, Canavan disease, Cockayne syndrome, Corticobasal degeneration, Creutzfeldt-Jakob disease, Huntington's disease, Kennedy's disease, Krabbe disease, Lewy body dementia, Machado-Joseph disease, multiple sclerosis, multiple system atrophy, Pelizaeus-Merzbacher disease, Pick's disease, primary lateral sclerosis, Refsum's disease, Sandhoff disease, Schilder's disease, Spielmeyer-Vogt-Sjogren-Batten disease, spinocerebellar ataxia, spinal muscular atrophy, Steele-Richardson-Olszewski disease, Tay-Sachs, transmissible spongiform encephalopathies (TSE), or Tabes dorsalis.

21. The method of claim 18, wherein the soluble epoxide hydrolase-related disease is inflammation.

22. The method of claim 21, wherein the inflammation is neuroinflammation, asthma, chronic obstructive pulmonary disorder (COPD), chronic bronchitis, cystic fibrosis, atherosclerosis, post-angioplasty, restenosis, coronary artery diseases, angina, rheumatoid arthritis, osteoarthritis, dermatitis, eczematous dermatitis, psoriasis, post transplantation late and chronic solid organ rejection, systemic lupus erythematosis, dermatomyositis, polymyositis, Sjogren's syndrome, polymyalgia rheumatica, temporal arteritis, Behcet's disease, Guillain Barre syndrome, Wegener's granulomatosus, polyarteritis nodosa, an inflammatory neuropathy, vasculitis, an inflammatory disorder of adipose tissue, Kaposi's sarcoma, or a smooth muscle cell proliferative disorder.

23. The method of claim 18, wherein the soluble epoxide hydrolase-related disease is hypertension, adult respiratory distress syndrome, end stage renal disease, heart failure, renal failure, liver failure, cardiac fibrosis, renal fibrosis, ischemic limb disease, intermittent claudication, endothelial dysfunction, erectile dysfunction, Raynaud's disease, a diabetic vasculopathy, an atherothrombotic disorder, or a metabolic disorder.

24. The method of claim 18, further comprising administering a second compound, biomolecule, or composition.

25. The method of claim 24, wherein the second compound, biomolecule, or composition is an anti-inflammatory agent.

26. The method of claim 25, wherein the anti-inflammatory agent is epoxyeicosatrienoic acid.

27. The method of claim 18, further comprising selecting a subject having an amyloid- and/or tau-biomarker or further comprising selecting an elderly subject.

28. (canceled)

29. The method of claim 18, wherein the step of administering the compound or the pharmaceutical composition is performed orally, intraperitoneally, sublingually, subcutaneously, intravenously, or any clinically acceptable administration route.

30. A method of reducing amyloid beta plaque formation in a subject, comprising:
administering to the subject an effective amount of a compound of claim 1.

31. A method of inhibiting soluble epoxide hydrolase in a cell, comprising:
contacting a cell with an effective amount of a compound of claim 1.

32. (canceled)
